(19) United States
(12) Patent Application Publication
(10) Pub. No.: US 2024/0350522 A1
(43) Pub. Date: Oct. 24, 2024

(54) HYGROMYCIN A FOR TREATMENT OF DISEASES AND INFECTIONS

(71) Applicant: FLIGHTPATH BIOSCIENCES, INC., Birmingham, MI (US)

(72) Inventors: Matthew Charles TINDALL, Berkeley, CA (US); Kim LEWIS, Berkeley, CA (US)

(73) Assignee: FLIGHTPATH BIOSCIENCES, INC., Birmingham, MI (US)

(21) Appl. No.: 18/693,393

(22) PCT Filed: Sep. 23, 2022
(86) PCT No.: PCT/US2022/076947

§ 371 Date: Mar. 19, 2024

Related U.S. Application Data
(60) Provisional application No. 63/247,927, filed on Sep. 24, 2021.
(60) Provisional application No. 63/247,928, filed on Sep. 24, 2021.
(60) Provisional application No. 63/247,929, filed on Sep. 24, 2021.

(51) Int. Cl. A61K 31/7048; A61P 31/04

(52) U.S. Cl. CPC A61K 31/7048; A61P 31/04

(57) ABSTRACT

The present disclosure provides therapeutic agents e.g., hygromycin A for use in the treatment, prevention, or management of diseases. The diseases can be caused by microbes of Fusobacterium or Treponema spp. The therapeutic agents of the disclosure can be used to inhibit the growth of the microbes described herein.

### CROSS REFERENCE TO RELATED APPLICATIONS

This application claims benefit of priority under 35 U.S.C. 119(e) to U.S. Provisional Application No. 63/247,927 filed on Sep. 24, 2021; U.S. Provisional Application No. 63/247,928 filed on Sep. 24, 2021; and U.S. Provisional Application No. 63/247,929 filed on Sep. 24, 2021, the contents of each of which are herein incorporated by reference in their entirety.

### BACKGROUND

Hygromycin A (also known as homomycin or totomycin) is a modified cinnamic acid antibiotic isolated from Streptomyces hygroscopicus. Initial studies demonstrated that hygromycin A had a broad spectrum of activity against gram-positive and gram-negative bacteria. Hygromycin acts by inhibiting ribosomal peptidyl transferase activity. Hygromycin A also blocks the binding of either chloramphenicol or lincomycin to the ribosomes.

### SUMMARY

The present disclosure provides therapeutic agents for inhibiting growth of a microbe. The therapeutic agent can be hygromycin A. The microbe can be Treponema spp., Streptococcus spp., Fusobacterium spp. Parvimonas spp., or Porphyromonas spp. The Treponema spp. can be Treponema denticola, Treponema pallidum or Treponema carateum. The Treponema pallidum can be Treponema pallidum pallidum, Treponema pallidum pertenue, or Treponema pallidum endemicum. The Fusobacterium spp. can be Fusobacterium nucleatum, including F. nucleatum animalis, F. nucleatum vincentii, F. nucleatum nucleatum, F. nucleatum polymorphum, F. nucleatum fusiforme, or F. nucleatum periodonticum. The Parvimonas spp. can be Parvimonas micra. The Porphyromonas spp. can be Porphyromonas gingivalis. In some embodiments, the concentration of hygromycin A is from about 0.01 μg/ml to about 100 μg/ml. In some embodiments, the concentration of hygromycin A is from about 0.01 μg/ml to about 1.0 μg/ml. In some embodiments, the concentration of hygromycin A can be 0.05 μg/ml. In some embodiments, the concentration of hygromycin A can be 0.1 μg/ml. In some embodiments, the concentration of hygromycin A can be 0.2 μg/ml. In some embodiments, the concentration of hygromycin A can be 0.5 μg/ml. In some embodiments, the concentration of hygromycin A can be 1.0 μg/ml. In some embodiments, the concentration of hygromycin A can be 2 μg/ml. In some embodiments, the concentration of hygromycin A can be 5 μg/ml.

The present disclosure provides therapeutic agents and methods related to oral microbes and the treatment, prevention and/or management of diseases associated with the oral microbes.

In some embodiments, the present disclosure provides a method for inhibiting growth of oral microbes.

The present disclosure provides methods for inhibiting or reducing the growth of oral microbes. in a subject. Such methods can include, contacting the subject with at least one therapeutic agent, such as, but not limited to hygromycin A.

Methods of the disclosure can include contacting oral microbes with at least one therapeutic agent. Contacting the oral microbes with the therapeutic agent can inhibit the growth of the oral microbes. In some embodiments, the therapeutic agent can be hygromycin A. The oral microbe can be Treponema denticola, Fusobacterium nucleatum, Parvimonas micra, or Porphyromonas gingivalis.

In some embodiments, the concentration of hygromycin A is from about 0.01 μg/ml to about 100 μg/ml. In some embodiments, the concentration of hygromycin A is from about 0.01 μg/ml to about 1.0 μg/ml. In some embodiments, the concentration of hygromycin A can be 0.05 μg/ml. In some embodiments, the concentration of hygromycin A can be 0.1 μg/ml. In some embodiments, the concentration of hygromycin A can be 0.2 μg/ml. In some embodiments, the concentration of hygromycin A can be 0.5 μg/ml. In some embodiments, the concentration of hygromycin A can be 1.0 μg/ml. In some embodiments, the concentration of hygromycin A can be 2 μg/ml. In some embodiments, the concentration of hygromycin A can be 5 μg/ml.

Also provided herein is a method for treating or preventing a disease preventing a disease associated with an oral microbe in a subject. The disease can be an oral disease, a systemic disease, a cancer or a veterinary disease. In some embodiments, the oral disease can be periodontitis or gingivitis. In some embodiments, the disease can be cancer, for example, colorectal cancer, gastric cancer or esophageal cancer.

The present disclosure provides therapeutic agents and methods related to F. nucleatum and the treatment, prevention and/or management of diseases associated with F. nucleatum.

In some embodiments, the present disclosure provides methods for inhibiting the growth of Fusobacterium nucleatum (F. nucleatum). Such methods can include contacting F. nucleatum with at least one therapeutic agent such that the growth of F. nucleatum is inhibited. The therapeutic agent can be hygromycin A. Also provided herein are methods for inhibiting or reducing the growth of Fusobacterium nucleatum.

In some embodiments, the concentration of hygromycin A can be from about 1 μg/ml to about 100 μg/ml. In some embodiments, the concentration of hygromycin A can be 5 μg/ml. In some embodiments, the concentration of hygromycin A can be 10 μg/ml. In some embodiments, the concentration of hygromycin A can be 20 μg/ml. In some embodiments, the concentration of hygromycin A can be 40 μg/ml.

The F. nucleatum can be one or more sub species, including F. nucleatum animalis, F. nucleatum vincentii, F. nucleatum nucleatum, F. nucleatum polymorphum, F. nucleatum fusiforme, or F. nucleatum periodonticum.

The present disclosure provides a method for treating or preventing a disease in a subject, the method comprising contacting the subject with at least one therapeutic agent. In some embodiments, the disease can be a cancer, a gastrointestinal disorder or an oral disease.

In some embodiments, the disease can be associated with a F. nucleatum infection. In some embodiments, the disease can be cancer, for example, colorectal cancer, or oral cancer. In some embodiments, the disease can be gastrointestinal disease, such as, inflammatory bowel disease, Crohn's disease, ulcerative colitis, or colorectal cancer. In some embodiments, the disease can be an oral disease, for example, periodontal disease. The periodontal disease can be localized aggressive periodontitis, generalized aggressive periodontitis, pulp necrosis or periapical periodontitis. In some embodiments, the disease can be oral cancer.

The present disclosure provides therapeutic agents and methods related to treponematoses and the treatment, prevention and/or management of diseases associated with Treponema pallidum infection.

Provided herein are methods for inhibiting the growth of Treponema by contacting Treponema with at least one therapeutic agent. Also provided herein are methods of reducing Treponema in a subject. Contacting Treponema or the subject with hygromycin A can inhibit the growth of Treponema. In some embodiments, the therapeutic agent can be hygromycin A. The Treponema can be Treponema pallidum or Treponema carateum.

In some embodiments, the concentration of hygromycin A can be from about 0.01 μg/ml to about 100 μg/ml.

In some embodiments, the concentration of hygromycin A can be from about from about 0.01 μg/ml to about 10 μg/ml.

In some embodiments, the concentration of hygromycin A can be 0.06 μg/ml. In some embodiments, the concentration of hygromycin A can be 0.12 μg/ml. In some embodiments, the concentration of hygromycin A can be 0.24 μg/ml. In some embodiments, the concentration of hygromycin A can be 0.48 μg/ml. In some embodiments, the concentration of hygromycin A can be 0.96 μg/ml. In some embodiments, the concentration of hygromycin A can be 1.92 μg/ml. In some embodiments, the concentration of hygromycin A can be 3.84 μg/ml.

The Treponema pallidum can be Treponema pallidum pallidum, Treponema pallidum pertenue, or Treponema pallidum endemicum.

The present disclosure also provides for a method for treating or preventing a treponematoses in a subject. Such methods can include contacting the subject with at least one therapeutic agent. The therapeutic agent can be hygromycin A. The treponematoses can be associated with Treponema pallidum or Treponema carateum infection. In some embodiments, the treponematoses can be syphilis. In some embodiments, the treponematoses can be yaws. In some embodiments, the treponematoses can be bejel. In some embodiments, the treponematoses can be pinta.

### DETAILED DESCRIPTION

### I. Introduction

With the advancement of microbial detection technologies, an increasing number of previously overlooked microorganisms have been discovered to play important roles in human diseases. A paradigm shift has also been made in understanding the key role of the microbiome in health and disease.

### Hygromycin A

In the last two decades, the impact of antibiotics on gut microbiome has been studied. Broad-spectrum antibiotics such as ampicillin, doxycycline, amoxicillin and ceftriaxone have been shown to impact the gut microbiota, causing rapid and diminished levels of bacterial diversity and changes to the relative abundances of bacteria, leading to dysbiosis. The microbiome shapes the immune system during development and contributes to maintaining a healthy gastrointestinal tract and preventing cardiovascular, neurological and autoimmune diseases. Accordingly, there is a need in the art for therapeutic agents, and new treatments and/or preventative measures against, diseases associated with bacteria that have minimal to no impact on gut microbiome.

Hygromycin A is a fermentation-derived natural product, first isolated from Streptomyces hygroscopicus in 1953. Hygromycin A was considered a broad-spectrum antibiotic due to activity reported against certain acid-fast bacteria as well as certain gram-positive bacteria and gram-negative bacteria (see U.S. Pat. No. 3,100,176). Hygromycin A only demonstrated modest effects against bacteria and was not pursued commercially.

In the present disclosure inventors demonstrate that Hygromycin A is effective against certain oral treponemes, Fusobacterium spp. and treponematoses causing organisms, while not affecting the beneficial bacteria of the oral cavity and/or gut. Examples of bacteria that are beneficial in the oral cavity and/or gut include, but are not limited to, Streptococcus oralis, Streptococcus parasanguinis, Bifidobacterium longum, Bacteroides nordii, Bacteroides cellulosilyticus, Streptococcus sanguinis, Parabacteroides merdae, Lactobacillus reuteri, Bacteroides fragilis, Blautia producta, Bacteroides ovatus, Bacteroides vulgatus, Bacteroides eggerthii, Enterococcus faecalis, Enterobacter cloacae, and/or Bacteroides xylanisolvens. Notably, oral treponemes and Fusobacterium can cause diseases in the mouth and gastrointestinal tract where the importance of microbiomes has previously been demonstrated. The present disclosure thus provides therapeutic agents that have the potential to target disease causing bacteria without affecting the microbiome. In an embodiment, the hygromycin A inhibits the growth of or kills the disease-causing bacteria but does not inhibit the growth of or kill at least one species of beneficial oral or gut bacteria.

### Overview of Oral Treponemes

Oral treponemes, along with over 600 other bacterial species, exist as part of a polymicrobial biofilm accreted to the tooth surface in the gingival crevice. Treponemes play a role in the etiology of several chronic diseases of humans including periodontal diseases including chronic periodontitis, acute necrotizing ulcerative gingivitis, endodontic infections and some acute dental abscesses. In addition, treponemes have been implicated in the development of chronic diseases of domestic animals, including periodontal diseases of dogs, bovine digital dermatitis of dairy cattle, and contagious ovine digital dermatitis.

Periodontal disease, also called gum disease, is a common affliction among adults caused by oral bacteria growth. Whereas complex microflora exists in healthy gingival plaque posing little or no health risk, periodontal lesions can form and become dominated by proteolytic Gram-negative anaerobes and spirochetes, which are associated with severe and refractory periodontal conditions. Spirochetes disrupt intercellular junctions, invade underlying tissue, and give rise to destructive host responses in advanced periodontal disease. The genus Treponema includes more than 60 phylotypes of oral spirochetes, of which Treponema denticola is the most cultivable, and is implicated in the etiology of periodontal disease. Novel therapeutics and methods are needed to treat oral infections, including infections implicated in periodontal disease, and for treating Treponema infection.

Among treponemes, Treponema denticola (also referred to herein as T. denticola) is one of the most widely studied oral microbes. Treponema denticola is a Gram-negative, obligate anaerobic, motile and highly proteolytic spirochete bacterium. It is one of four species of oral spirochetes to be reliably cultured, the others being Treponema pectinovorum, Treponema socranskii and Treponema vincentii. T. denticola dwells in a complex and diverse microbial community within the oral cavity and is highly specialized to survive in this environment.

Given the widespread role of oral microbes in disease, there remains a need to develop therapeutic agents that inhibit, reduce and/or kill oral microbes, particularly Treponema denticola. The present disclosure provides therapeutic agents, such as, but not limited to, hygromycin A for diseases associated with oral microbes.

Overview of Fusobacterium nucleatum

With the advancement of microbial detection technologies, an increasing number of previously overlooked microorganisms have been discovered to play important roles in human diseases. A paradigm shift has also been made in understanding the role of the microbiome in health and disease. Fusobacterium nucleatum (herein referred to as F. nucleatum), a Gram-negative anaerobe, one such emerging pathogen.

F. nucleatum is ubiquitous in the oral cavity, absent or infrequently detected elsewhere in the body under normal conditions. Under disease conditions, F. nucleatum has been detected in extra-oral sites. F. nucleatum is a heterogeneous species with several proposed subspecies (ss), i.e., ss animalis, ss fusiforme, ss nucleatum, ss polymorphum, ss periodonticum and ss vincentii, whose prevalence in disease vary.

Fusobacterium nucleatum (F. nucleatum) is a Gram-negative obligate anaerobe bacterium in the oral cavity and plays a role in several oral diseases, including periodontitis and gingivitis. Recently, several studies have reported that the level of F. nucleatum is significantly elevated in human colorectal adenomas and carcinomas compared to that in adjacent normal tissue. A causal role for F. nucleatum in the pathogenesis of colorectal cancer has also been demonstrated. There remains a need to identify therapeutic agents that target F. nucleatum and/or for the treatment of F. nucleatum associated diseases. Fusobacterium nucleatum is an invasive, adherent and pro-inflammatory anaerobic bacterium. It is common in dental plaque and there is a well-established association between F. nucleatum and periodontitis. Anecdotally, F. nucleatum has been implicated in cerebral abscesses and pericarditis and it is one of the Fusobacterium species implicated in Lemierre's syndrome, a rare form of thrombophlebitis. Various Fusobacteria, including F. nucleatum, have been implicated in acute appendicitis, where they have been found by immunohistochemistry (IHC) as epithelial and submucosal infiltrates that correlate positively with severity of disease. When isolated from human intestinal biopsy material, F. nucleatum has been found to be more readily culturable from patients with gastrointestinal (GI) disease than healthy controls, and the strains grown from inflamed biopsy tissue appeared to exhibit a more invasive phenotype.

Given the widespread role of F. nucleatum in disease, there remains a need to develop therapeutic agents that inhibit, reduce and/or kill F. nucleatum. The present disclosure provides therapeutic agents, such as, but not limited to, hygromycin A for treatment of diseases associated with F. nucleatum.

### Overview of Treponematoses

The human treponematoses comprise venereal syphilis and the endemic treponematoses including yaws, bejel, and Pinta. The etiological agents of these diseases are Gram-negative bacteria that belong to the order Spirochaetales, family Spirochaetaceae, and genus Treponema. Syphilis, yaws, and bejel spirochetes were originally classified as separate species but are now considered to be subspecies of Treponema pallidum (T. pallidum subsp. pallidum, T. pallidum subsp. pertenue, and T. pallidum subsp. endemicum, respectively). The lack of availability of an isolate of the agent of pinta has precluded genetic analyses of this organism, and it retains its separate name, T. carateum.

All human treponematoses share similarities in pathogenesis and natural history. All are transmitted by direct contact with infectious lesions and are chronic infections that manifest in multiple stages involving the skin. All except pinta can progress to cause serious and destructive lesions of skin, bone, and cartilage. As in venereal syphilis, the clinical manifestations of the endemic treponematoses are commonly divided into an early stage (encompassing primary and secondary manifestations) and a late stage. Early-stage lesions are highly infectious and can persist for weeks to months, or even years, following appearance. Once the early manifestations spontaneously regress due to the host's immune response against the pathogen, the patient enters a state of latency that in many cases lasts for a lifetime. In a relatively small percentage of cases, however, the infection can progress from latency to tertiary disease, characterized by destruction of tissues.

Treponematoses have not yet been eradicated. Distinctive features have been identified in terms of age of acquisition, mode of transmission, and capacity for invasion of the central nervous system. In 2012, the World Health Organization (WHO) set a goal to eradicate treponematoses. Successful eradication requires therapeutic strategies targeting the causative Treponema. The present disclosure provides therapeutic agents for the treatment, prevention and/or management of Treponematoses.

### II. Therapeutic Agents

In some embodiments, the present disclosure provides therapeutic agents for the treatment of diseases such as, but not limited to, oral microbe associated diseases. In some embodiments, the treatment of the disease in a subject can involve administering at least one therapeutic agent. In some embodiments, the therapeutic agent can be hygromycin A.

Hygromycin A (Hyg A) is a product of Streptomyces hygroscopicus first isolated in 1953. It has a unique structure consisting of a furanose, cinnamic acid and aminocyclitol moiety. It has a relatively broad antimicrobial spectrum. Hygromycin A has the structure shown here:

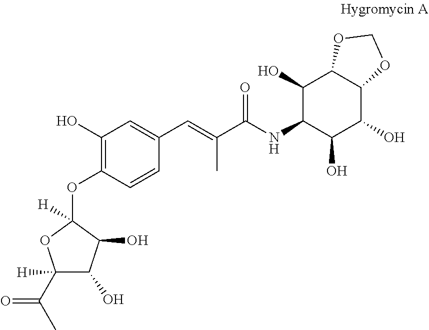 [SMILES: CC(=O)[C@H]1O[C@@H](Oc2ccc(/C=C(\C)C(=O)N[C@@H]3[C@H](O)[C@@H](O)[C@H]4OCO[C@H]4[C@@H]3O)cc2O)[C@@H](O)[C@@H]1O]

### Combinations

In some embodiments, a combination of therapeutic agents can be utilized. The present disclosure provides two, three, four, five or more therapeutic agents in a combinatorial format. Combinations can be administered concurrently, sequentially and/or serially. In some embodiments, each therapeutic agent in a combination can be formulated as separate pharmaceutical formulations. In some embodiments, the therapeutic agents in a combination can be prepared as single pharmaceutical formulation.

For purposes of the present disclosure, “in combination”, refers to providing two or more therapeutic agents either separately or together, where the two therapeutic agents are administered as part of an appropriate dose regimen designed to obtain the benefit of the combination therapy. Thus, the two therapeutic agents can be administered either as part of the same pharmaceutical composition or in separate pharmaceutical compositions. The first therapeutic agent can be administered prior to, at the same time as, or subsequent to administration of the second therapeutic agent, or in some combination thereof. Where the one therapeutic agent is administered to the subject at repeated intervals, e.g., during a standard course of treatment, the second therapeutic agent can be administered prior to, at the same time as, or subsequent to, each administration of the first therapeutic agent, or some combination thereof, or at different intervals in relation to therapy with the first therapeutic agent, or in a single dose prior to, at any time during, or subsequent to the course of treatment with the first therapeutic agent.

Combinations of therapeutic agents of the present disclosure can include hygromycin A and a second therapeutic agent. In some embodiments, the second therapeutic agent can be a therapeutic agent utilized to treat a Fusobacterium nucleatum infection.

Combinations of therapeutic agents of the present disclosure can include hygromycin A and a second therapeutic agent used to treat inflammation, destruction of connective tissues, periodontal ligament, and alveolar bone resorption, and ultimately tooth loss in chronic periodontal infection. In some embodiments, the second therapeutic agent can be enoxacin and/or a bisphosphonate derivative of enoxacin (bis-enoxacin).

Combinations of therapeutic agents of the present disclosure can include hygromycin A and a second therapeutic agent. In some embodiments, the second therapeutic agent can be a therapeutic agent utilized to treat treponematoses.

In some embodiments, the therapeutic agents can be combined with one or more agents used in the treatment of syphilis. In some embodiments, the therapeutic agents of the present disclosure can be combined with penicillin.

In some embodiments, the therapeutic agents can be combined with one or more agents used in the treatment of yaws. In some embodiments, the therapeutic agents of the present disclosure can be combined with azithromycin and/or benzathine benzylpenicillin.

In some embodiments, the therapeutic agents can be combined with one or more agents used in the treatment of Bejel or Pinta. In some embodiments, the therapeutic agents of the present disclosure can be combined with doxycycline and/or benzathine benzylpenicillin.

### III. Methods of Use

The present disclosure provides methods of use related to the therapeutic agents described herein. In some embodiments, the methods can include a method of reducing the growth of bacteria e.g., an oral microbe. As used herein, the term “oral microbe” is applied to any microorganism that inhabits the oral cavity. In some embodiments, the oral microbes can be present in the oral cavity of healthy individuals. In some embodiments, the presence of oral microbes in the oral cavity can be associated with a disease or pathological state.

### Oral and Periodontal Disease

The therapeutic agents of the disclosure can exhibit potency against oral microbes, and therefore have the potential to treat, and/or prevent an infection, or kill and/or inhibit the growth of an oral microbe. In some embodiments, the animal can be a human. In some embodiments, the spirochete infection can be treated and/or prevented, or the spirochete can be killed, or its growth is inhibited, through oral administration of the therapeutic agent of the disclosure. As a non-limiting example, the spirochete infection can be treated and/or prevented, or the spirochete can be killed, or its growth is inhibited through intravenous administration of the therapeutic agent of the disclosure.

In some embodiments, the oral microbe can be a Treponema spp., a Streptococcus spp., a Fusobacterium spp., and/or a Parvimonas spp.

In some embodiments, the therapeutic agents of the present disclosure can be used for treating, preventing, protecting against and/or managing a disease caused by Treponema such as, but not limited to T. pallidum, T. denticola, T. vincentii, T. carateum, and/or T. phagedenis or clinical isolates or strains thereof.

In some embodiments, the therapeutic agents of the present disclosure can be used for treating, preventing, protecting against and/or managing a disease caused by an oral microbe e.g., Treponema such as, but not limited to T. pallidum, T. denticola, T. vincentii, T. carateum, and/or T. phagedenis or clinical isolates or strains thereof.

In some embodiments, the oral microbe can be Treponema denticola or a strain or a clinical isolate thereof, such as T. denticola (strain: ATCC35405), T. denticola (strain: ATCC35404), T. denticola (strain: ATCC33520), T. denticola (strain: ATCC33521), T. denticola (strain: OTK), T. denticola (strain: H1-T), T. denticola (strain: H-22), T. denticola (strain: MYR-T), T. denticola (strain: US-Trep), T. denticola (strain: ASLM), T. denticola (strain: AL-2), T. denticola (strain: SP21), T. denticola (strain: SP23), T. denticola (strain: SP32), T. denticola (strain: SP33), T. denticola (strain: SP37), and/or T. denticola (strain: SP44).

In some embodiments, the therapeutic agents of the disclosure can be used to treat, prevent or manage a disease caused by, or associated with an oral microbe such as, but not limited to, Fusobacterium nucleatum (e.g., any strain of F. nucleatum animalis, ATCC 25586, ATCC5 51191, ATCC 10953, ATCC 23726, CTI-2, CTI-3, CTI-7, EAV0002, 7-1, FA2+, 7-33 C1), Veillonella parvula (e.g., ATCC 10790), Actinomyces odontolyticus (e.g., ATCC 17982), Neisseria mucosa, Parvimonas micra (e.g., ATCC 33270), Porphyromonas gingivalis (e.g., ATCC 33277), Tannerella forsythia, Capnocytophaga, Peptostreptococcus, and/or Eikenella.

In some embodiments, oral microbes can include Porphyromonas gingivalis, Fusobacterium nucleatum, Prevotella intermedia, Prevotella loescheii, Triponema denticola (Treponema denticola), Porphyromonas endodontalis, Peptococcus anaerobius, Micros prevotii, Eubasterium limosum, Centipedia perio Centipedia periodontii, Selenomonas arterimidis, Fusobacterium periodonticum, Eubacterium spp., Bacteroides spp., Actinomyces viscosos, Streptococcus mutans, and/or Streptococcus sobrinus.

In some embodiments, administering a therapeutic agent described herein to a subject infected with an oral microbe or having a disease caused or compounded/exacerbated by the oral microbe, inhibits or reduces replication of the oral microbe by at least 20% to 25%, at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In some embodiments, administering a therapeutic agent described herein to a subject (in some embodiments, an animal model) infected with an oral microbe inhibits or reduces replication of the infectious agent by at least 1.5 fold, 2 fold, 2.5 fold, 3 fold, 4 fold, 5 fold, 8 fold, 10 fold, 15 fold, 20 fold, or 2 to 5 fold, 2 to 10 fold, 5 to 10 fold, or 5 to 20 fold relative to a negative control as determined using an assay described herein or others known to one of skill in the art.

In some aspects, the therapeutic agent can be used to treat or prevent periodontal diseases. Periodontal diseases are mainly the result of infections and inflammation of the gums and bone that surround and support the teeth. In its early stage, called gingivitis, the gums can become swollen and red, and they can bleed. In its more serious form, called periodontitis, the gums can pull away from the tooth, bone can be lost, and the teeth can loosen or even fall out. In some embodiments, the therapeutic agents of the disclosure can be used to treat gingivitis. In some embodiments, the therapeutic agents of the disclosure can be used to treat periodontitis.

In some embodiments, the therapeutic agents of the disclosure can be used to treat, manage or prevent one or more symptoms associated with periodontal disease, such as, but not limited to, bad breath or bad taste, red or swollen gums, tender or bleeding gums, painful chewing, loose teeth, sensitive teeth, gums that have pulled away from teeth.

In some embodiments, the therapeutic agents of the disclosure can be used to treat, manage or prevent chronic periodontitis. Chronic periodontitis is a polymicrobial disease that results from the overgrowth of a limited number of bacterial species that are normal members of the oral microbiota. It is widely accepted that Treponema denticola, Porphyromonas gingivalis, and Tannerella forsythia form a bacterial consortium, often referred to as the ‘Red Complex’, that is strongly associated with the clinical progression of chronic periodontitis. The unifying features of the Red Complex bacteria are their extracellular proteolytic activity, their complex anaerobic fermentations of amino acids, production of toxic metabolites, and outer membrane (or sheath) vesicles.

In some embodiments, the therapeutic agents of the disclosure can be used to treat, prevent, or manage gingival disease. In some embodiments, the gingival disease can be caused by or correlated with the infection by or the presence of oral microbes.

In some embodiments, the therapeutic agents of the disclosure can be used to treat, prevent, or manage chronic periodontitis. In some embodiments, the chronic periodontitis can be caused by or correlated with the infection by or the presence of oral microbes.

In some embodiments, the therapeutic agents of the disclosure can be used to treat, prevent, or manage aggressive periodontitis. In some embodiments, the aggressive periodontitis can be caused by or correlated with the infection by or the presence of oral microbes.

In some embodiments, the therapeutic agents of the disclosure can be used to treat, prevent, or manage abscesses of the periodontium. In some embodiments, the abscesses of the periodontium can be caused by or correlated with the infection by or the presence of oral microbes.

In some embodiments, the therapeutic agents of the disclosure can be used to treat, prevent, or manage periodontitis associated with endodontic lesion. In some embodiments, the periodontitis associated with endodontic lesion of the periodontium can be caused by or correlated with the infection by or the presence of oral microbes.

In some embodiments, the therapeutic agents of the disclosure can be used to treat Stage I of periodontitis characterized by 1-2 mm interdental clinical attachment loss. In some embodiments, the therapeutic agents of the disclosure can be used to treat Stage II of periodontitis characterized by 3-4 mm interdental clinical attachment loss. In some embodiments, the therapeutic agents of the disclosure can be used to treat Stage III or Stage IV of periodontitis characterized by >5 mm interdental clinical attachment loss.

In some embodiments, the concentration of hygromycin A can be about, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1, 2, 3, 4, 5, 6, 7, 8, 9, 10, 15, 20, 25, 30, 35, 40, 45, 50, 55, 60, 65, 70, 75, 80, 85, 90, 95, 100 μg/ml or more.

Fusobacterium nucleatum Associated Diseases

In some embodiments, the therapeutic agents of the disclosure can be used to treat, prevent, or manage Fusobacterium nucleatum associated oral infections in subjects.

F. nucleatum is one of the most abundant species in the oral cavity, in both diseased and healthy individuals. In some embodiments, the therapeutic agents of the disclosure can be used to treat, prevent or manage Fusobacterium nucleatum associated periodontal diseases such as, but not limited to, gingivitis and the advanced irreversible forms of periodontitis including chronic periodontitis, localized aggressive periodontitis and generalized aggressive periodontitis. Therapeutic agents of the disclosure can be used to treat, prevent or manage endodontic infections such as pulp necrosis and periapical periodontitis which are frequently associated with F. nucleatum infections.

In one embodiment, the oral disease is oral cancer.

The prevalence of F. nucleatum has been correlated with an increase in with the severity of disease, progression of inflammation and pocket depth. Among the five subspecies, ss fusiforme and ss vincentii are more frequently associated with healthy state while ss nucleatum with disease state. In addition to the periodontal sites, F. nucleatum is detected in saliva, and is increased in number in patients with gingivitis and periodontitis, compared to the healthy controls. Serum antibody titers to F. nucleatum have been reported to be elevated in diseased patients.

F. nucleatum is an invasive bacterium that causes acute oral and gastrointestinal infections and can act as a pro-inflammatory agent. In some embodiments, the therapeutic agents of the present disclosure can be used to treat, prevent or manage Fusobacterium nucleatum associated diseases. As used herein, the term “Fusobacterium nucleatum associated diseases” can be used to refer to a disease or a condition that is caused by and/or associated with infection by one or more species of Fusobacterium nucleatum.

In some embodiments, the F. nucleatum associated disease can be an inflammatory condition, such as, but not limited to, sinusitis, endocarditis, septic arthritis, tonsillitis and abscesses of the brain, skin and/or liver.

At least 6 different sub species of F. nucleatum have been described and include F. nucleatum animalis, F. nucleatum fusiforme, F. nucleatum nucleatum, F. nucleatum polymorphum, F. nucleatum periodonticum and F. nucleatum vincentii. In some embodiments, the F. nucleatum can be a strain associated with a disease state. In some embodiments, the strain can be a F. nucleatum animalis subspecies strain such as, but not limited to, 7/1 (or 71), CRC 7/3 JVN3C1 (or CRC 7_3JVN3C1), and/or 218A8. In some embodiments, the strain can be a F. nucleatum vincentii subspecies strain such as, but not limited to, 215A9, CC53 and/or 3/1/36A2 (EAV018). In some embodiments, the strain can be a F. nucleatum nucleatum subspecies strain such as, but not limited to, ATCC25586T, 203L34 and/or 2/3 FMU 1 (2_3 FMU 1). In some embodiments, the strain can be a F. nucleatum polymorphum strain such as, but not limited to, 203L28, 203L29, and/or 13/3C (13_3C EAV005 or EAVG 005). In some embodiments, the strain can be a F. nucleatum fusiforme strain such as, but not limited to 203C15, 203L25, and/or 203L30. In some embodiments, the strain can be a F. nucleatum periodonticum strain, such as, but not limited to, 2/1/31 or (2_1_31 EAV015 or EAVG 015), 3/1/7B (31_7B or EAVG 011) and/or 209B32.

In some embodiments, the present disclosure provides methods for inhibiting the growth of Fusobacterium species e.g., Fusobacterium nucleatum. The growth of the F. nucleatum species can be inhibited in vitro (e.g., in a cell, or a tissue sample from a subject) or in vivo in a subject. In some aspects, the growth of the F. nucleatum can be inhibited by about 10%, 20%, 30%, 40%, 50%, 60%, 70%, 80% and/or 90%. In some embodiments, the growth of the bacteria is inhibited by 5-15%, 10-20%, 15-25%, 20-30%, 25-35%, 30-40%, 35-45%, 40-50%, 45-55%, 50-60%, 55-65%, 60-70%, 65-75%, 70-80%, 75-85%, and/or 90-100%.

In some embodiments, administering a therapeutic agent described herein to a subject infected with a Fusobacterium or having a disease caused or compounded/exacerbated by Fusobacterium, inhibits or reduces replication of the Fusobacterium by at least 20% to 25%, at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In some embodiments, administering a therapeutic agent described herein to a subject (in some embodiments, an animal model) infected with Fusobacterium inhibits or reduces replication of the infectious agent by at least 1.5 fold, 2 fold, 2.5 fold, 3 fold, 4 fold, 5 fold, 8 fold, 10 fold, 15 fold, 20 fold, or 2 to 5 fold, 2 to 10 fold, 5 to 10 fold, or 5 to 20 fold relative to a negative control as determined using an assay described herein or others known to one of skill in the art.

The present disclosure provides therapeutic agents and methods related to F. nucleatum and the treatment, prevention and/or management of diseases associated with F. nucleatum.

In some embodiments, the present disclosure provides methods for inhibiting the growth of Fusobacterium nucleatum (F. nucleatum). Such methods can include contacting F. nucleatum with at least one therapeutic agent such that the growth of F. nucleatum is inhibited. The therapeutic agent can be hygromycin A. Also provided herein are methods for inhibiting or reducing the growth of Fusobacterium nucleatum.

In some embodiments, the concentration of hygromycin A can be from about 1 μg/ml to about 100 μg/ml. In some embodiments, the concentration of hygromycin A can be 5 μg/ml. In some embodiments, the concentration of hygromycin A can be 10 μg/ml. In some embodiments, the concentration of hygromycin A can be 20 μg/ml. In some embodiments, the concentration of hygromycin A can be 40 μg/ml.

In some embodiments, the concentration of hygromycin A can be about, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1, 2, 3, 4, 5, 6, 7, 8, 9, 10, 15, 20, 25, 30, 35, 40, 45, 50, 55, 60, 65, 70, 75, 80, 85, 90, 95, 100 μg/ml or more.

The F. nucleatum can be one or more sub species, including F. nucleatum animalis, F. nucleatum vincentii, F. nucleatum nucleatum, F. nucleatum polymorphum, F. nucleatum fusiforme, or F. nucleatum periodonticum.

The present disclosure provides a method for treating or preventing a disease in a subject, the method comprising contacting the subject with at least one therapeutic agent. In some embodiments, the disease can be a cancer, a gastrointestinal disorder or an oral disease.

In some embodiments, the disease can be associated with a F. nucleatum infection. In some embodiments, the disease can be cancer, for example, colorectal cancer, or oral cancer. In some embodiments, the disease can be gastrointestinal disease, such as, inflammatory bowel disease, Crohn's disease, ulcerative colitis, or colorectal cancer. In some embodiments, the disease can be an oral disease, for example, periodontal disease. The periodontal disease can be localized aggressive periodontitis, generalized aggressive periodontitis, pulp necrosis or periapical periodontitis. In some embodiments, the disease can be oral cancer.

In some embodiments, the concentration of hygromycin A can be about, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1, 2, 3, 4, 5, 6, 7, 8, 9, 10, 15, 20, 25, 30, 35, 40, 45, 50, 55, 60, 65, 70, 75, 80, 85, 90, 95, 100 μg/ml or more.

### Systemic Diseases

The presence of microbes and their metabolic by-products in the mouth can modulate the immune response beyond the oral cavity, thus promoting the development of systemic conditions. A growing body of literature suggests that there is a link between oral microbes and systemic diseases.

Therapeutic agents of the disclosure can be used to treat, manage or prevent systemic diseases associated with oral microbes such as, but not limited to cardiovascular disease, gastrointestinal disease, colorectal cancer, diabetes and insulin resistance, Alzheimer's disease, as well as respiratory tract infection and adverse pregnancy outcomes.

Oral microbes such as oral pathogens can promote development of non-oral disease directly or indirectly. About 30 abundant species in the oral cavity, mainly Gram-negative anaerobic bacteria, are known to produce endotoxins, which could directly contribute to systemic disease. Migration of oral pathogens to the blood stream could also occur in some cases, such as following surgical procedures. Bacterial accumulation on the teeth due to poor dental hygiene and/or environmental factors induces a host inflammatory response, which can result in periodontitis and bone loss but could also be harmful to the organism systemically.

In some embodiments, the therapeutic agents of the present disclosure can be used to treat, prevent, or manage bacteremia. Tissue trauma, flossing, dental procedures or even chewing can induce breakage of blood vessels in the close proximity of dental plaques containing oral microbes, which can introduce the oral microbes into the systemic bloodstream resulting in bacteremia.

In some embodiments, the therapeutic agents of the disclosure can be used to treat, prevent, or manage cardiovascular disease. In some embodiments, the cardiovascular diseases can be caused by or correlated with the infection by or the presence of oral microbes. Bacterial DNA of species such as, Treponema denticola, Actinobacillus actinomycetemcomitans, Tannerella forsythia, Eikenella corrodens, Fusobacterium nucleatum and Campylobacter rectus have been identified in atheromatous and atherosclerotic plaques retrieved by surgery.

In some embodiments, the therapeutic agents of the disclosure can be used to treat, prevent, or manage respiratory tract infection. In some embodiments, the respiratory tract infection can be caused by or correlated with the infection by or the presence of oral microbes. In some embodiments, the therapeutic agents of the disclosure can be used to treat, manage or prevent Lemierre's syndrome, which can be caused by Fusobacterium nucleatum and Fusobacterium necrophorum.

In some embodiments, the therapeutic agents of the disclosure can be used to treat, prevent, or manage pneumonia. In some embodiments, the pneumonia can be caused by or correlated with the infection by or the presence of oral microbes.

In some embodiments, the therapeutic agents of the disclosure can be used to treat, prevent, or manage diabetes. In some embodiments, diabetes can be caused by or correlated with the infection by or the presence of oral microbes. Chronic infection during periodontitis can lead to exacerbated and dysregulated inflammatory responses, which can result in poor metabolic control of blood sugar and increased insulin requirements. Conversely, diabetes can also lead to different complications such as poor wound healing, retinopathy, nephropathy, neuropathy, macrovascular disease and periodontitis.

In some embodiments, the therapeutic agents of the disclosure can be used to treat, prevent, or manage Alzheimer's disease. In some embodiments, Alzheimer's disease can be caused by or correlated with the infection by or the presence of oral microbes, e.g., Fusobacterium nucleatum.

In some embodiments, the concentration of hygromycin A can be about, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1, 2, 3, 4, 5, 6, 7, 8, 9, 10, 15, 20, 25, 30, 35, 40, 45, 50, 55, 60, 65, 70, 75, 80, 85, 90, 95, 100 μg/ml or more.

### Cancer

In some embodiments, the therapeutic agents of the present disclosure are used for treating, protecting against, and/or managing cancer. In some embodiments, the therapeutic agents of the disclosure are used for treating, preventing or managing a cancer associated with one or more oral microbes.

In some embodiments, the therapeutic agents of the disclosure are used for treating, preventing or managing a cancer caused by Fusobacterium. In some embodiments, the cancer can be oral, colorectal or esophageal cancer. As a non-limiting example, the colorectal or esophageal cancer can be caused by Fusobacterium species.

The subject can have cancer, can be suspected of having cancer, or can have a predisposition to cancer. The therapeutic agents or pharmaceutically acceptable salts thereof can be administered to the subject as a treatment for cancer and maintenance in all patients. The effect of the therapeutic agents or formulations described herein on proliferation of cancer cells can be detected by routine assays known in the art. Cancer cell lines on which such assays can be performed are well known to those of skill in the art.

Oral microbes including Fusobacterium nucleatum, Porphyromonas gingivalis, Treponema denticola, Aggregatibacter actinomycetemcomitans, and Tannerella forsythia have been receiving increasing interest in the context of cancer etiology. Periodontal pathogens can be associated with pancreatic and oral cancers. In some embodiments, the therapeutic agents of the disclosure can be used to inhibit, reduce or limit the growth of one or more oral microbes associated with cancer. Oral microbes can contribute to carcinogenesis via different mechanisms such as inhibition of apoptosis, activation of cell proliferation, promotion of cellular invasion, induction of chronic inflammation, and production of carcinogens. As a non-limiting example, hygromycin A can be used to inhibit the immunosuppressive effect of Fusobacterium nucleatum by promoting polarization of macrophages through a TLR4-dependent mechanism in colorectal cancer.

In some embodiments, the therapeutic agents of the disclosure can be used to treat, manage or prevent colorectal cancers, gastric cancer and/or esophageal cancers.

The present disclosure also provides methods of administering a therapeutic agent, as described herein, to a subject. The subject can have cancer, can be suspected of having cancer, or can have a predisposition to cancer. The compounds or pharmaceutically acceptable salts thereof are administered to the subject as a treatment for cancer and maintenance in all patients. The effect of the compositions described herein on proliferation of cancer cells can be detected by routine assays known in the art. Cancer cell lines on which such assays can be performed are well known to those of skill in the art.

Cancers and related disorders that can be treated, protected against, or managed using the therapeutic agents and methods described herein include, but are not limited to, the following: leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, and chronic lymphocytic leukemia, hairy cell leukemia; polycythemia vera; lymphomas such as but not limited to Hodgkin's disease, and non-Hodgkin's disease; multiple myelomas such as but not limited to smoldering multiple myeloma, non-secretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, and synovial sarcoma; brain tumors including but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including but not limited to, pheochromocytoma and adrenocortical carcinoma; thyroid cancer such as but not limited to papillary or follicular thyroid cancer, medullary thyroid cancer and anaplastic thyroid cancer; pancreatic cancer, including but not limited to, insulinoma, gastrinoma, glucagonoma, vipoma, somatostatin-secreting tumor, and carcinoid or islet cell tumor; pituitary cancers including but not limited to, Cushing's disease, prolactin-secreting tumor, acromegaly, and diabetes insipidus; eye cancers including but not limited to, ocular melanoma such as iris melanoma, choroidal melanoma, and ciliary body melanoma, and retinoblastoma; vaginal cancers, including but not limited to, squamous cell carcinoma, adenocarcinoma, and melanoma; vulvar cancer, including but not limited to, squamous cell carcinoma, melanoma, adenocarcinoma, basal cell carcinoma, sarcoma, and Paget's disease; cervical cancers including but not limited to, squamous cell carcinoma, and adenocarcinoma; uterine cancers including but not limited to, endometrial carcinoma and uterine sarcoma; ovarian cancers including but not limited to, ovarian epithelial carcinoma, borderline tumor, germ cell tumor, and stromal tumor; esophageal cancers including but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers including but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including but not limited to hepatocellular carcinoma and hepatoblastoma; gallbladder cancers including but not limited to, adenocarcinoma; cholangiocarcinomas including but not limited to, papillary, nodular, and diffuse; lung cancers including but not limited to, non-small cell lung cancer, squamous cell carcinoma (epidermoid carcinoma), adenocarcinoma, large-cell carcinoma and small-cell lung cancer; testicular cancers including but not limited to, germinal tumor, seminoma, anaplastic, embryonal carcinoma, teratoma carcinoma, choriocarcinoma (yolk-sac tumor); prostate cancers including but not limited to, adenocarcinoma, leiomyosarcoma, and rhabdomyosarcoma; penal cancers; oral cancers including but not limited to, squamous cell carcinoma; basal cancers; salivary gland cancers including but not limited to, adenocarcinoma, mucoepidermoid carcinoma, and adenoid cystic carcinoma; pharynx cancers including but not limited to, squamous cell cancer, and verrucous; skin cancers including but not limited to, basal cell carcinoma, squamous cell carcinoma and melanoma, and superficial spreading melanoma, nodular melanoma, lentigo malignant melanoma, acral lentiginous melanoma; kidney cancers including but not limited to, renal cell cancer, renal cancer, adenocarcinoma, hypernephroma, fibrosarcoma, and transitional cell cancer (renal pelvis and/or ureter); Wilms' tumor; bladder cancers including but not limited to, transitional cell carcinoma, squamous cell cancer, adenocarcinoma, and carcinosarcoma. In addition, cancers include myxosarcoma, osteogenic sarcoma, endotheliosarcoma, lymphangioendothelioma sarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas.

In some embodiments, the cancer is benign, e.g., polyps and benign lesions. In other embodiments, the cancer is metastatic. The compositions described herein can be used in the treatment of pre-malignant as well as malignant conditions. Pre-malignant conditions include hyperplasia, metaplasia, and dysplasia. Treatment of malignant conditions includes the treatment of primary as well as metastatic tumors. In some embodiments the cancer is melanoma, colon cancer, lung cancer, breast cancer, prostate cancer, cervical cancer, brain cancer, pancreatic cancer, or renal cancer, T-cell acute lymphocytic leukemia (ALL), a B-cell acute lymphocytic leukemia, a lymphoblastic leukemia, a B-cell chronic lymphocytic leukemia or a B-cell non-Hodgkin's lymphoma, rhabdomyosarcoma, neuroblastoma, Ewing sarcoma, gastric cancer, hepatoma.

In some embodiments, the concentration of hygromycin A can be about, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1, 2, 3, 4, 5, 6, 7, 8, 9, 10, 15, 20, 25, 30, 35, 40, 45, 50, 55, 60, 65, 70, 75, 80, 85, 90, 95, 100 μg/ml or more.

### Gastrointestinal Disease

In some embodiments, the therapeutic agents can be used to treat, prevent, or manage Fusobacterium nucleatum associated gastrointestinal diseases or disorders such as, but not limited to colorectal cancer (CRC), inflammatory bowel disease (IBD) and appendicitis.

Cancers of the gastrointestinal tract represent a significant percentage of all cancer related deaths, and include gastric cancer, colorectal and esophageal cancers. Colorectal carcinoma (CRC) is the second leading cause of cancer deaths, responsible for approximately 655,000 deaths per year worldwide. CRC is also one of the first and best genetically characterized cancers in which specific somatic mutations on oncogenes and tumor suppressor genes associated with progression from adenomatous lesions (polyps) to invasive carcinoma have been identified. Inflammation has been recognized as a risk factor for CRC. In some embodiments, the therapeutic agents of the disclosure can be used to treat, prevent, or manage colorectal cancer. In some embodiments, the therapeutic agents of the disclosure can be used to treat, prevent or manage Fusobacterium nucleatum associated colorectal cancer. The CRC can be caused by or linked to F. nucleatum infection.

In some embodiments, the therapeutic agents of the disclosure can be used to treat, prevent or manage IBD. In some embodiments, the therapeutic agents of the disclosure can be used to treat, prevent or manage Fusobacterium nucleatum associated IBD. IBD has been recognized as a risk factor for CRC. F. nucleatum strains isolated from inflamed tissues of the IBD patients are more invasive than those from the normal tissues. Several studies have reported association of F. nucleatum in appendicitis. Co-occurrence of F. nucleatum with other oral taxa has been observed.

In some embodiments, the therapeutic agents of the disclosure can be used to treat, prevent or manage Crohn's disease. In some embodiments, the therapeutic agents of the disclosure can be used to treat, prevent or manage Fusobacterium nucleatum associated Crohn's disease.

In some embodiments, the therapeutic agents of the disclosure can be used to treat, prevent or manage ulcerative colitis. In some embodiments, the therapeutic agents of the disclosure can be used to treat, prevent, or manage Fusobacterium nucleatum associated ulcerative colitis.

In some embodiments, the concentration of hygromycin A can be about, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1, 2, 3, 4, 5, 6, 7, 8, 9, 10, 15, 20, 25, 30, 35, 40, 45, 50, 55, 60, 65, 70, 75, 80, 85, 90, 95, 100 μg/ml or more.

### Veterinary Diseases

In some embodiments, therapeutic agents can be used in the treatment, prevention or management of one or more disease in animals (also referred to herein as veterinary diseases). In some embodiments, the veterinary diseases can be caused by bacteria, such as, but not limited to, Treponema, Fusobacterium, Bacteroides, Campylobacter, Mycoplasma and Porphyromonas.

In some embodiments, the therapeutic agents of the disclosure can be used to treat, prevent or manage digital dermatitis (DD). DD is a foot disease that causes lameness in cattle. It is characterized by an inflammatory dermatitis of the digital skin, most commonly on the plantar aspect of the interdigital cleft. In some embodiments, the therapeutic agents can be used to inhibit the growth of bacterial species associated with DD. As a non-limiting example, DD can be caused by Treponema spp. In some embodiments, the Treponema can be Treponema phagedenis, T. medium, and T. pedis.

In some embodiments, therapeutic agents can be used in the treatment, prevention or management of one or more periodontal disease in dogs.

In some embodiments, the therapeutic agents of the disclosure can be used in the treatment, prevention or management of Treponeme associated hoof disease. Treponema associated hoof disease (TAHD) in elk, is a debilitating and progressive condition, which shares many similarities to bovine digital dermatitis and contagious ovine digital dermatitis.

In some embodiments, the concentration of hygromycin A can be about, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1, 2, 3, 4, 5, 6, 7, 8, 9, 10, 15, 20, 25, 30, 35, 40, 45, 50, 55, 60, 65, 70, 75, 80, 85, 90, 95, 100 μg/ml or more.

### Treponematoses

The present disclosure provides therapeutic agents and methods related to treponematoses and the treatment, prevention and/or management of diseases associated with Treponema pallidum infection.

Provided herein are methods for inhibiting the growth of Treponema by contacting Treponema with at least one therapeutic agent. Also provided herein are methods of reducing Treponema in a subject. Contacting Treponema or the subject with hygromycin A can inhibit the growth of Treponema. In some embodiments, the therapeutic agent can be hygromycin A. The Treponema can be Treponema pallidum or Treponema carateum.

In some embodiments, the concentration of hygromycin A can be from about 0.01 μg/ml to about 100 μg/ml. In some embodiments, the concentration of hygromycin A can be about, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1, 2, 3, 4, 5, 6, 7, 8, 9, 10, 15, 20, 25, 30, 35, 40, 45, 50, 55, 60, 65, 70, 75, 80, 85, 90, 95, 100 μg/ml or more.

In some embodiments, the concentration of hygromycin A can be from about from about 0.01 μg/ml to about 10 μg/ml.

In some embodiments, the concentration of hygromycin A can be 0.06 μg/ml. In some embodiments, the concentration of hygromycin A can be 0.12 μg/ml. In some embodiments, the concentration of hygromycin A can be 0.24 μg/ml. In some embodiments, the concentration of hygromycin A can be 0.48 μg/ml. In some embodiments, the concentration of hygromycin A can be 0.96 μg/ml. In some embodiments, the concentration of hygromycin A can be 1.92 μg/ml. In some embodiments, the concentration of hygromycin A can be 3.84 μg/ml.

The Treponema pallidum can be Treponema pallidum pallidum, Treponema pallidum pertenue, or Treponema pallidum endemicum.

The present disclosure also provides for a method for treating or preventing a treponematoses in a subject. Such methods can include contacting the subject with at least one therapeutic agent. The therapeutic agent can be hygromycin A. The treponematoses can be associated with Treponema pallidum or Treponema carateum infection. In some embodiments, the treponematoses can be syphilis. In some embodiments, the treponematoses can be yaws. In some embodiments, the treponematoses can be bejel. In some embodiments, the treponematoses can be pinta.

The present disclosure provides methods of use related to the therapeutic agents described herein. In some embodiments, the methods can include a method of reducing the growth of bacteria such as Treponema pallidum.

In some embodiments, the therapeutic agents of the disclosure can be used to kill or inhibit the growth of one or more sub species of Treponema pallidum. Subspecies of Treponema include, but are not limited to, Treponema pallidum pallidum, Treponema carateum, Treponema pallidum pertenue, and Treponema pallidum endemicum.

In some embodiments, the present disclosure provides methods for inhibiting the growth of Treponema pallidum. The growth of the T. pallidum species can be inhibited in vitro (e.g., in a cell or a co-culture system, or a tissue sample from a subject) or in vivo in a subject. In some aspects, the growth of the T. pallidum can be inhibited by about 10%, 20%, 30%, 40%, 50%, 60%, 70%, 80% and/or 90%. In some embodiments, the growth of the bacteria can be inhibited by 5-15%, 10-20%, 15-25%, 20-30%, 25-35%, 30-40%, 35-45%, 40-50%, 45-55%, 50-60%, 55-65%, 60-70%, 65-75%, 70-80%, 75-85%, and/or 90-100%.

In some embodiments, administering a therapeutic agent described herein to a subject infected with a Treponema pallidum or having a disease caused or compounded/exacerbated by Treponema pallidum, inhibits or reduces replication of Treponema pallidum by at least 20% to 25%, at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% relative to a negative control as determined using an assay described herein or others known to one of skill in the art. In some embodiments, administering a therapeutic agent described herein to a subject (in some embodiments, an animal model) infected with Treponema pallidum inhibits or reduces replication of the infectious agent by at least 1.5 fold, 2 fold, 2.5 fold, 3 fold, 4 fold, 5 fold, 8 fold, 10 fold, 15 fold, 20 fold, or 2 to 5 fold, 2 to 10 fold, 5 to 10 fold, or 5 to 20 fold relative to a negative control as determined using an assay described herein or others known to one of skill in the art.

In some embodiments, the therapeutic agents of the disclosure can be used to treat, prevent and/or manage venereal treponematoses or syphilis.

In some embodiments, the therapeutic agents of the disclosure can be used to treat, prevent and/or manage endemic treponematoses or non-venereal treponematoses. Important differences between endemic treponematoses and syphilis relate to the target population, mode of transmission, and the tendency for systemic involvement. Endemic treponematoses predominantly affect children in the poor rural communities whereas syphilis is a universal disease. Endemic treponematoses can include bejel, yaws and pinta.

### Syphilis

In some embodiments, therapeutic agents of the disclosure can be used to treat Syphilis. Syphilis is a systemic disease caused by T. pallidum pallidum. The disease has been divided into stages on the basis of clinical findings, which guide treatment and follow-up. In some embodiments, the therapeutic agents of the disclosure can be used to treat primary syphilis. Primary syphilis classically presents as a single painless ulcer or chancre at the site of infection but can also present with multiple, atypical, or painful lesions. In some embodiments, the therapeutic agents of the present disclosure can be used to treat, prevent or manage secondary syphilis which is characterized by skin rash, mucocutaneous lesions, and lymphadenopathy. In some embodiments, the therapeutic agents of the disclosure can be used to treat, manage and/or prevent tertiary syphilis which can present with cardiac involvement, gummatous lesions, tabes dorsalis, and general paresis.

In some embodiments, the therapeutic agents of the disclosure can be used to treat latent infections. Latent infections (i.e., those lacking clinical manifestations) are detected by serologic testing. Latent syphilis acquired within the preceding year is referred to as early latent syphilis; all other cases of latent syphilis are classified as late latent syphilis or latent syphilis of unknown duration.

In some embodiments, the therapeutic agents of the disclosure can be used to treat, prevent or manage central nervous system (CNS) associated symptoms syphilis. T. pallidum can infect the CNS, at any stage of syphilis and result in neurosyphilis. Early neurologic clinical manifestations or syphilitic meningitis (e.g., cranial nerve dysfunction, meningitis, meningovascular syphilis, stroke, and acute altered mental status) are usually present within the first few months or years of infection. Late neurologic manifestations (e.g., tabes dorsalis and general paresis) occur 10 to >30 years after infection.

In some embodiments, the therapeutic agents of the disclosure can be used to treat, prevent or manage ocular syphilis or otosyphilis. Infection of the visual system (ocular syphilis) or auditory system (otosyphilis) can occur at any stage of syphilis but is commonly identified during the early stages and can present with or without additional CNS involvement. Ocular syphilis often presents as panuveitis but can involve structures in both the anterior and posterior segment of the eye, including conjunctivitis, anterior uveitis, posterior interstitial keratitis, optic neuropathy, and retinal vasculitis. Ocular syphilis can result in permanent vision loss. Otosyphilis typically presents with cochleo-vestibular symptoms, including tinnitus, vertigo, and sensorineural hearing loss. Hearing loss can be unilateral or bilateral, have a sudden onset, and progress rapidly. Otosyphilis can result in permanent hearing loss.

In some embodiments, the concentration of hygromycin A can be about, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1, 2, 3, 4, 5, 6, 7, 8, 9, 10, 15, 20, 25, 30, 35, 40, 45, 50, 55, 60, 65, 70, 75, 80, 85, 90, 95, 100 μg/ml or more.

### Yaws

In some embodiments, therapeutic agents of the disclosure can be used to treat, prevent or manage yaws. Transmission of yaws, caused by T. pallidum subsp. pertenue, occurs by direct skin contact with an infectious lesion and is facilitated by breaks in the skin of traumatic or other etiology (e.g., scratches or scabies). In some embodiments, the therapeutic agents of the disclosure can be used to treat one or more stages of the disease. The primary stage of the disease appears after a variable incubation period (approximately 21 days) as a solitary erythematous papule that can grow into a papilloma of 2 to 5 cm in diameter by peripheral extension or by coalescing with satellite papules. The lesion is not painful but can be pruritic, and it is typically covered by a crust that hides an ulcer with raised dark margins and an erythematous moist center, overall resembling a raspberry (hence the African and French names). In some embodiments, the therapeutic agents of the disclosure can be used to treat, prevent and/or manage the primary lesion, which is often found on the lower extremities. The lesion is highly contagious and can persist for weeks or months before healing spontaneously, often leaving a hypopigmented or depressed area delimited by a dark border. At this stage, regional lymphadenopathy and arthralgia can also occur. In some embodiments, the therapeutic agents of the disclosure can be used to treat lymphadenopathy and/or arthralgia associated with yaws. In the majority of cases, the primary lesion heals spontaneously before the onset of yaws' secondary manifestations, whose appearance is due to the pathogen's systemic dissemination during early infection. In some embodiments, the therapeutic agents of the disclosure can be used to treat systemic dissemination associated with yaws. In some embodiments, the therapeutic agents of the disclosure can be used to treat, manage or prevent secondary manifestations associated with yaws. Secondary manifestations can include condylomata lata in moist crevices such as the axilla and groin and a measles-like eruption. Hyperkeratotic plaques on palms and soles are also common at this stage. In some embodiments, the therapeutic agents of the disclosure can be used to treat periostitis and osteitis which can affect the bones of the upper and lower limbs (tibia, fibula, and forearm) and the proximal phalanges of fingers and toes, resulting in bone pain and digital swelling. In some embodiments, the therapeutic agents of the disclosure can be used to treat latent disease. The secondary lesions heal spontaneously within weeks or months, and the patient enters the latent stage of the infection, which can be recognized only through serological tests and will, unless treated, last for a lifetime. Recurrences (generally one or two) of secondary manifestations might be seen up to 5 years after the initial infection, although relapses after 10 years have been reported.

In some embodiments, the therapeutic agents of the disclosure can be used to treat, prevent or manage tertiary yaws. Approximately 10% of untreated patients will develop tertiary yaws, characterized by subcutaneous gummatous nodules, chronic periostitis that can cause apparent bowing of the tibia (i.e., saber shin), and destructive processes leading to saddle nose and perforation/collapse of the palate and nasal septum (i.e., gangosa). Bilateral hypertrophic periostitis of the paranasal maxilla and nasal bridge causes the clinical manifestation known as goundou.

### Bejel

In some embodiments, the therapeutic agents of the disclosure can be used to treat Bejel. Bejel is the Arabic name for endemic (or nonvenereal) syphilis caused by T. pallidum subsp. endemicum. In some embodiments, the therapeutic agents of the disclosure can be used to treat acute infection, which is observed in in children between 2 and 15 years of age in dry, arid climates. Although the mode of transmission has not been adequately studied, it is believed to occur through mucosal and skin contact or the sharing of eating utensils or drinking vessels.

In contrast to the other treponematoses, bejel's primary lesion is often unobserved. When seen, however, it appears as a small and painless mucous papule or ulcer that develops in the oral cavity or nasopharynx. In some embodiments, the therapeutic agents of the disclosure can be used to treat primary and/or secondary lesions associated with bejel. Secondary lesions are very similar to those of venereal syphilis and can manifest as mucous patches on the oral mucosa, tonsils, tongue, lips, and nasopharynx. Split papules at the labial commissures (angular stomatitis, as in yaws patients), non-itchy skin eruptions, generalized lymphadenopathy, and laryngitis are common manifestations. Secondary skin lesions include condylomata lata in intertriginous body areas, comparable to those in yaws and syphilis. Maculopapular or papulosquamous lesions, as well as a nonpruritic generalized papular rash, can be observed in a minority of patients with bejel. As in yaws, osteitis and periostitis of the long bones and hands can occur, causing nocturnal bone pain. Secondary manifestations heal in 6 to 9 months, and the disease enters latency. In some embodiments, the therapeutic agents of the present disclosure can be used to treat tertiary stage of bejel. The tertiary stage might manifest earlier than for yaws (6 months to several years) but, as in yaws, is characterized by gummatous lesions of the skin, mucosa, and bone that can progress to destructive ulcers. Skin lesions resolve in time and leave characteristic depigmented scars surrounded by hyperpigmentation.

In some embodiments, the concentration of hygromycin A can be about, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1, 2, 3, 4, 5, 6, 7, 8, 9, 10, 15, 20, 25, 30, 35, 40, 45, 50, 55, 60, 65, 70, 75, 80, 85, 90, 95, 100 μg/ml or more.

### Pinta

In some embodiments, the therapeutic agents of the present disclosure can be used to treat, prevent and/or manage Pinta (also known as mal de pinto, enfermedad azul and carate or cute). In some embodiments, the therapeutic agents of the disclosure can be used to treat Pinta caused by Treponema carateum, and is regarded as the mildest of the treponematoses, in that its lesions are limited to the skin and there is no evidence of systemic involvement or vertical transmission. The disease is found focally in tropical Central and South America. Because no laboratory strain of this pathogen is currently available for studies, T. carateum is the least characterized of the agents of the human treponematoses and is still classified independently from the other T. pallidum subspecies, whose genetic and antigenic relatedness has been experimentally demonstrated. The mechanism of transmission is unknown, although repeated skin-to-skin contact appears to be the most plausible. Young adults (≤15 years old) with chronic skin lesions are considered to be the disease's main reservoir. Pinta also appears to be transmitted to infants from their mothers by close contact. In some embodiments, the therapeutic agents of the disclosure can be used to treat early or late stages of the Pinta. The primary lesion manifests as a papule or an erythemato-squamous plaque on exposed parts of the body after an incubation period of 1 week to 2 months. Satellite lesions can be present. With time, the papules increase in size and coalesce to form patches with a pale center. After months, many of the patches become hypochromic or acquire a light-blue/grayish pigmentation, with the color more marked at the center of the lesion. The initial lesions can either heal, leaving a slightly pigmented or hypochromic area, or persist for years and become indistinguishable from the secondary lesions. Regional lymphadenopathy is common at this stage. After months or years, small disseminated secondary lesions (called pintids) can appear in the form of scaly papules that will again enlarge and coalesce in psoriasiform plaques. These plaques might become hypo- or hyperchromic, as well as erythematous or desquamative. Different types of pintids might be present at the same time in the same individual. This stage usually lasts 2 to 4 years, during which some patches will heal, and others will persist and enlarge. The late stage usually develops 2 to 4 years after initial infection and is characterized by the appearance of pigmentary changes, skin atrophy, and hyperkeratosis.

In some embodiments, the concentration of hygromycin A can be about, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1, 2, 3, 4, 5, 6, 7, 8, 9, 10, 15, 20, 25, 30, 35, 40, 45, 50, 55, 60, 65, 70, 75, 80, 85, 90, 95, 100 μg/ml or more.

### Other Indications

In some embodiments, the therapeutic agents of the disclosure can be used to treat, prevent or manage Fusobacterium nucleatum associated infections and abscesses including infections of the head and neck (Lemierre's syndrome, acute and chronic mastoiditis, chronic otitis and sinusitis, tonsillitis, peritonsillar and retropharyngeal abscesses, postanginal cervical lymphadenitis, periodontitis), brain, lungs, abdomen, pelvis, bones, joints, and blood.

In some embodiments, the therapeutic agents of the disclosure can be used to treat, prevent or manage cardiovascular diseases. F. nucleatum has been implicated in cardiovascular diseases (CVD). It is frequently detected in the atherosclerotic plaques and is also one of the most common periodontal pathogens detected in ruptured cerebral aneurysm.

In some embodiments, the therapeutic agents of the disclosure can be used to treat, prevent or manage cerebral aneurysm. In some embodiments, the therapeutic agents of the disclosure can be used to treat, prevent or manage Fusobacterium nucleatum associated cerebral aneurysm.

In some embodiments, the therapeutic agents of the disclosure can be used to treat, prevent or manage Lemierre's syndrome. In some embodiments, the therapeutic agents of the disclosure can be used to treat, prevent or manage Fusobacterium nucleatum associated Lemierre's syndrome.

In some embodiments, the therapeutic agents of the disclosure can be used to treat, prevent or manage respiratory tract infections. In some embodiments, the therapeutic agents of the disclosure can be used to treat, prevent or manage Fusobacterium nucleatum associated respiratory tract infections.

In some embodiments, the therapeutic agents of the disclosure can be used to treat, prevent or manage organ abscesses. In some embodiments, the therapeutic agents of the disclosure can be used to treat, prevent or manage Fusobacterium nucleatum associated organ abscesses.

In some embodiments, the therapeutic agents of the disclosure can be used to treat, prevent or manage rheumatoid arthritis. In some embodiments, the therapeutic agents of the disclosure can be used to treat, prevent or manage Fusobacterium nucleatum associated rheumatoid arthritis.

In some embodiments, the therapeutic agents of the disclosure can be used to treat, prevent or manage Alzheimer's disease. In some embodiments, the therapeutic agents of the disclosure can be used to treat, prevent or manage Fusobacterium nucleatum associated Alzheimer's disease.

### Co-Infections and Co-Morbidities

In some embodiments, the therapeutic agents of the disclosure can be used to treat, prevent, or manage a subject who has additional bacterial infections. In some embodiments, the subject can have a disease associated with Tannerella forsythia, Porphyromonas gingivalis and Streptococcus.

In some embodiments, the therapeutic agents of the disclosure can be used to treat, prevent, or manage a subject who has co-morbidities. As a non-limiting example, the co-morbidity can be diabetes (type 1 or type 2), heart disease, and/or hypertension, SARS-CoV-2 infection (e.g., COVID-19).

### IV. Formulations

In some embodiments, therapeutic agents can be administered to humans, human patients or subjects. For the purposes of the present disclosure, the phrase “active ingredient” generally refers to the therapeutic agents to be delivered as described herein.

Although the descriptions of formulations provided herein are principally directed to formulations which are suitable for administration to humans, it will be understood by the skilled artisan that such therapeutic agents are generally suitable for administration to any other animal, e.g., to non-human animals, e.g., non-human mammals. Modification of formulations suitable for administration to humans in order to render the therapeutic agents suitable for administration to various animals is well understood, and the ordinarily skilled veterinary pharmacologist can design and/or perform such modification with merely ordinary, if any, experimentation. Subjects to which administration of the formulations is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the therapeutic agents described herein can be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A formulation in accordance with the disclosure can be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a formulation in accordance with the disclosure will vary, depending upon the identity, size, and/or condition of the subject treated and further depending upon the route by which the formulation is to be administered. By way of example, the formulation can include between 0.1% and 100%, e.g., between 0.5 and 50%, between 1-30%, between 5-80%, or, in some embodiments, at least 20%, at least 40%, at least 60%, or at least 80% (w/w) active ingredient.

The therapeutic agents of the present disclosure can be formulated using one or more excipients to: (1) increase stability; (2) permit the sustained or delayed release; (3) alter the biodistribution; (4) alter the release profile of the therapeutic agents in vivo. Non-limiting examples of the excipients include any and all solvents, dispersion media, diluents, or other liquid vehicles, dispersion or suspension aids, surface active agents, isotonic agents, thickening or emulsifying agents, and preservatives. Excipients of the present disclosure can also include, without limitation, lipidoids, liposomes, lipid nanoparticles, polymers, lipoplexes, core-shell nanoparticles, peptides, proteins, hyaluronidase, nanoparticle mimics and combinations thereof.

In some embodiments, pharmaceutical compositions or formulations of the disclosure can be adapted to deliver a prescribed dosage of one or more therapeutic agents to a cell, a group of cells, an organ or tissue, an animal or a human. Methods of incorporating therapeutic agents into pharmaceutical preparations are widely known in the art. The determination of an appropriate prescribed dosage of a pharmacologically active compound to include in a pharmaceutical formulation in order to achieve a desired biological outcome is within the skill level of an ordinary practitioner of the art. The pharmaceutical formulation can include excipients, such as without limitation, binders, coating, disintegrants, fillers, diluents, flavors, colors, lubricants, glidants, preservatives, sorbents, sweeteners, conjugated linoleic acid (CLA), gelatin, beeswax, purified water, glycerol, any type of oil, including, without limitation, fish oil or soybean oil, or the like. Therapeutic agents and/or pharmaceutical formulations can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols. It will further be appreciated by an ordinary practitioner of the art that the term also encompasses those therapeutic agents and/or pharmaceutical formulations that contain an admixture of two or more pharmacologically active compounds, such compounds being administered, for example, as a combination therapy.

A pharmaceutical formulation in accordance with the present disclosure can be prepared, packaged, and/or sold in bulk, as a single unit dose, and/or as a plurality of single unit doses. As used herein, a “unit dose” refers to a discrete amount of the pharmaceutical formulation comprising a predetermined amount of therapeutic agent or other compounds. The amount of therapeutic agent can generally be equal to the dosage of therapeutic agent administered to a subject and/or a convenient fraction of such dosage including, but not limited to, one-half or one-third of such a dosage.

In some embodiments, the therapeutic agents of the disclosure can be formulated as a toothpaste, a mouth wash, a gum massage cream, an edible film, or floss.

### Excipients

Formulations can additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients can optionally be included in pharmaceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEENn®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC®F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives can include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

### V. Dosing and Administration

In some embodiments, therapeutic agents and/or pharmaceutical formulations that include therapeutic agents can be administered according to one or more administration routes. In some embodiments, administration is enteral (into the intestine), transdermal, intravenous bolus, intralesional (within or introduced directly to a localized lesion), intrapulmonary (within the lungs or its bronchi), diagnostic, intraocular (within the eye), transtympanic (across or through the tympanic cavity), intravesical infusion, sublingual, nasogastric (through the nose and into the stomach), spinal, intracartilaginous (within a cartilage), insufflation (snorting), rectal, intravascular (within a vessel or vessels), buccal (directed toward the cheek), dental (to a tooth or teeth), intratesticular (within the testicle), intratympanic (within the aurus media), percutaneous, intrathoracic (within the thorax), submucosal, cutaneous, epicutaneous (application onto the skin), dental intracornal, intramedullary (within the marrow cavity of a bone), intra-abdominal, epidural (into the dura matter), intramuscular (into a muscle), intralymphatic (within the lymph), iontophoresis (by means of electric current where ions of soluble salts migrate into the tissues of the body), subcutaneous (under the skin), intragastric (within the stomach), nasal administration (through the nose), transvaginal, intravenous drip, endosinusial, intraprostatic (within the prostate gland), soft tissue, intradural (within or beneath the dura), subconjunctival, oral (by way of the mouth), peridural, parenteral, intraduodenal (within the duodenum), intracisternal (within the cisterna magna cerebellomedularis), periodontal, periarticular, biliary perfusion, intracoronary (within the coronary arteries), intrathecal (within the cerebrospinal fluid at any level of the cerebrospinal axis), intrameningeal (within the meninges), intracavernous injection (into a pathologic cavity) intracavitary (into the base of the penis), intrabiliary, subarachnoid, intrabursal, ureteral (to the ureter), intratendinous (within a tendon), auricular (in or by way of the ear), intracardiac (into the heart), enema, intraepidermal (to the epidermis), intraventricular (within a ventricle), intramyocardial (within the myocardium), intratubular (within the tubules of an organ), vaginal, sublabial, intracorporus cavernosum (within the dilatable spaces of the corporus cavernosa of the penis), intradermal (into the skin itself), intravitreal (through the eye), perineural, cardiac perfusion, irrigation (to bathe or flush open wounds or body cavities), in ear drops, endotracheal, intraosseous infusion (into the bone marrow), caudal block, intrauterine, transtracheal (through the wall of the trachea), intra-articular, intracorneal (within the cornea), endocervical, extracorporeal, intraspinal (within the vertebral column), transmucosal (diffusion through a mucous membrane), topical, photopheresis, oropharyngeal (directly to the mouth and pharynx), occlusive dressing technique (topical route administration which is then covered by a dressing which occludes the area), transplacental (through or across the placenta), intrapericardial (within the pericardium), intraarterial (into an artery), interstitial, intracerebral (into the cerebrum), intracerebroventricular (into the cerebral ventricles), intrapleural (within the pleura), infiltration, intrabronchial, intrasinal (within the nasal or periorbital sinuses), intraductal (within a duct of a gland), intracaudal (within the cauda equine), nerve block, retrobulbar (behind the pons or behind the eyeball), intravenous (into a vein), intra-amniotic, conjunctival, intrasynovial (within the synovial cavity of a joint), gastroenteral, intraluminal (within a lumen of a tube), electro-osmosis, intraileal (within the distal portion of the small intestine), intraesophageal (to the esophagus), extra-amniotic administration, hemodialysis, intragingival (within the gingivae), intratumor (within a tumor), eye drops (onto the conjunctiva), laryngeal (directly upon the larynx), urethral (to the urethra), intravaginal administration, intraperitoneal (infusion or injection into the peritoneum), respiratory (within the respiratory tract by inhaling orally or nasally for local or systemic effect), intradiscal (within a disc), ophthalmic (to the external eye), and/or intraovarian (within the ovary).

In some embodiments, therapeutic agents and/or pharmaceutical formulations that include therapeutic agents can be administered by intraarticular administration, extracorporeal administration, intrabronchial administration, endocervical administration, endosinusial administration, endotracheal administration, enteral administration, epidural administration, intra-abdominal administration, intrabiliary administration, intrabursal administration, oropharyngeal administration, interstitial administration, intracardiac administration, intracartilaginous administration, intracaudal administration, intracavernous administration, intracerebral administration, intracorporus cavernosum, intracavitary administration, intracorneal administration, intracisternal administration, cranial administration, intracranial administration, intradermal administration, intralesional administration, intratympanic administration, intragingival administration, intraocular administration, intradiscal administration, intraductal administration, intraduodenal administration, ophthalmic administration, intradural administration, intraepidermal administration, intraesophageal administration, nasogastric administration, nasal administration, laryngeal administration, intraventricular administration, intragastric administration, intrahepatic administration, intraluminal administration, intravitreal administration, intravesicular administration, intralymphatic administration, intramammary administration, intramedullary administration, intrasinal administration, intrameningeal administration, intranodal administration, intraovarian administration, intraperitoneal administration, intrapleural administration, intraprostatic administration, intraluminal administration, intraspinal administration, intrasynovial administration, intratendinous administration, intratesticular administration, subconjunctival administration, intracerebroventricular administration, epicutaneous administration, intravenous administration, retrobulbar administration, periarticular administration, intrathoracic administration, subarachnoid administration, intratubular administration, periodontal administration, transtympanic administration, transtracheal administration, intratumor administration, vaginal administration, urethral administration, intrauterine administration, oral administration, gastroenteral administration, parenteral administration, sublingual administration, ureteral administration, percutaneous administration, peridural administration, transmucosal administration, perineural administration, transdermal administration, rectal administration, soft tissue administration, intraarterial administration, subcutaneous administration, topical administration, extra-amniotic administration, ear drops, or intravesical infusion.

Therapeutic agents and/or pharmaceutical formulations of the present disclosure can be administered orally but any suitable route of administration can be employed for providing a subject with an effective dosage of drugs of the chemical compositions described herein. For example, oral, rectal, topical, parenteral, ocular, pulmonary, nasal, and the like can be employed. Dosage forms include tablets, troches, dispersions, suspensions, solutions, capsules, creams, ointments, aerosols, and the like. In certain embodiments, it can be advantageous that the compositions described herein be administered orally.

Therapeutic agents and/or pharmaceutical formulations of the present disclosure can be administered in the conventional manner by any route where they are active. Administration can be systemic, parenteral, topical, or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration of the composition of the present disclosure (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

For administration by inhalation or intranasal, pharmaceutical formulation can be delivered in the form of an aerosol spray presentation from pressurized packs or nebulizers. The compounds can also be delivered in the form of a cream, liquid, spray, powder, or suppository. A metered dose of the formulation can be provided from a reservoir of the formulation. In addition, predetermined dosages can be provided, for example, suppository forms can be provided for insertion into the nose having a predetermined dosage. Kits can be provided, where prepared dosage forms and instructions for administering the dosages are included.

Suitable topical formulations for use in the present embodiments can also include transdermal devices, aerosols, creams, ointments, lotions, dusting powders, gels, and the like.

### Dosing

Therapeutic agents and/or pharmaceutical formulations described herein can be administered to a subject using any amount and any route of administration effective treating a disease, disorder, and/or condition. The exact amount required will vary from subject to subject, depending on the species, age, and general condition of the subject, the severity of the disease, the particular formulation, its mode of administration, its mode of activity, and the like.

In some embodiments, the therapeutic agents described herein can be provided to the subject at a concentration of from about 0.01 μg/ml to about 100 μg/ml. In some embodiments, the therapeutic agents can be provided to the subject at a concentration of from about 0.01 to about 0.05 μg/ml, from about 0.05 μg/ml to about 1.0 μg/ml, from about 1.0 μg/ml to about 10 μg/ml, from about 10 μg/ml to about 50 μg/ml, from about 50 μg/ml to about 100 μg/ml. In some embodiments, the therapeutic agents described herein can be provided to the subject at a concentration of from about 1 μg/ml to about 5 μg/ml, from about 5 μg/ml to about 10 μg/ml, from about 10 μg/ml to about 20 μg/ml, from about 20 μg/ml to about 30 μg/ml, from about 30 μg/ml to about 40 μg/ml, from about 40 μg/ml to about 50 μg/ml, from about 50 μg/ml to about 60 μg/ml, from about 60 μg/ml to about 70 μg/ml, from about 70 μg/ml to about 80 μg/ml, from about 80 μg/ml to about 90 μg/ml, from about 90 μg/ml to about 100 μg/ml.

In some embodiments, the concentration of hygromycin A can be about, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1, 2, 3, 4, 5, 6, 7, 8, 9, 10, 15, 20, 25, 30, 35, 40, 45, 50, 55, 60, 65, 70, 75, 80, 85, 90, 95, 100 μg/ml or more.

In some embodiments the therapeutic agents described herein can be provided to the subject at a concentration of 0.05 μg/ml.

In some embodiments the therapeutic agents described herein can be provided to the subject at a concentration of 0.06 μg/ml. In some embodiments the therapeutic agents described herein can be provided to the subject at a concentration of 0.12 μg/ml. In some embodiments the therapeutic agents described herein can be provided to the subject at a concentration of 0.24 μg/ml. In some embodiments the therapeutic agents described herein can be provided to the subject at a concentration of 0.48 μg/ml. In some embodiments the therapeutic agents described herein can be provided to the subject at a concentration of 0.96 μg/ml. In some embodiments the therapeutic agents described herein can be provided to the subject at a concentration of 1.92 μg/ml. In some embodiments the therapeutic agents described herein can be provided to the subject at a concentration of 3.84 μg/ml. In some embodiments the therapeutic agents described herein can be provided to the subject at a concentration of 0.1 μg/ml. In some embodiments the therapeutic agents described herein can be provided to the subject at a concentration of 0.2 μg/ml. In some embodiments the therapeutic agents described herein can be provided to the subject at a concentration of 0.5 μg/ml. In some embodiments the therapeutic agents described herein can be provided to the subject at a concentration of 1.0 μg/ml. In some embodiments the therapeutic agents described herein can be provided to the subject at a concentration of 2.0 μg/ml. In some embodiments the therapeutic agents described herein can be provided to the subject at a concentration of 5.0 μg/ml. In some embodiments the therapeutic agents described herein can be provided to the subject at a concentration of 10 μg/ml. In some embodiments the therapeutic agents described herein can be provided to the subject at a concentration of 20 μg/ml. In some embodiments the therapeutic agents described herein can be provided to the subject at a concentration of 40 μg/ml.

In some embodiments, therapeutic agents and/or pharmaceutical formulations of the present disclosure are provided in one or more doses and are administered one or more times to subjects. Some therapeutic agents and/or pharmaceutical formulations are provided in only a single administration. Some therapeutic agents and/or pharmaceutical formulations are provided according to a dosing schedule that include two or more administrations. Each administration can be at the same dose or can be different from a previous and/or subsequent dose. In some embodiments, subjects are provided an initial dose that is higher than subsequent doses (referred to herein as a “loading dose”). In some embodiments, doses are decreased over the course of administration. In some embodiments, dosing schedules include pharmaceutical formulation administration from about every 2 hours to about every 10 hours, from about every 4 hours to about every 20 hours, from about every 6 hours to about every 30 hours, from about every 8 hours to about every 40 hours, from about every 10 hours to about every 50 hours, from about every 12 hours to about every 60 hours, from about every 14 hours to about every 70 hours, from about every 16 hours to about every 80 hours, from about every 18 hours to about every 90 hours, from about every 20 hours to about every 100 hours, from about every 22 hours to about every 120 hours, from about every 24 hours to about every 132 hours, from about every 30 hours to about every 144 hours, from about every 36 hours to about every 156 hours, from about every 48 hours to about every 168 hours, from about every 2 days to about every 10 days, from about every 4 days to about every 15 days, from about every 6 days to about every 20 days, from about every 8 days to about every 25 days, from about every 10 days to about every 30 days, from about every 12 days to about every 35 days, from about every 14 days to about every 40 days, from about every 16 days to about every 45 days, from about every 18 days to about every 50 days, from about every 20 days to about every 55 days, from about every 22 days to about every 60 days, from about every 24 days to about every 65 days, from about every 30 days to about every 70 days, from about every 2 weeks to about every 8 weeks, from about every 3 weeks to about every 12 weeks, from about every 4 weeks to about every 16 weeks, from about every 5 weeks to about every 20 weeks, from about every 6 weeks to about every 24 weeks, from about every 7 weeks to about every 28 weeks, from about every 8 weeks to about every 32 weeks, from about every 9 weeks to about every 36 weeks, from about every 10 weeks to about every 40 weeks, from about every 11 weeks to about every 44 weeks, from about every 12 weeks to about every 48 weeks, from about every 14 weeks to about every 52 weeks, from about every 16 weeks to about every 56 weeks, from about every 20 weeks to about every 60 weeks, from about every 2 months to about every 6 months, from about every 3 months to about every 12 months, from about every 4 months to about every 18 months, from about every 5 months to about every 24 months, from about every 6 months to about every 30 months, from about every 7 months to about every 36 months, from about every 8 months to about every 42 months, from about every 9 months to about every 48 months, from about every 10 months to about every 54 months, from about every 11 months to about every 60 months, from about every 12 months to about every 66 months, from about 2 years to about 5 years, from about 3 years to about 10 years, from about 4 years to about 15 years, from about 5 years to about 20 years, from about 6 years to about 25 years, from about 7 years to about 30 years, from about 8 years to about 35 years, from about 9 years to about 40 years, from about 10 years to about 45 years, from about 15 years to about 50 years, or more than every 50 years.

The desired dosage can be delivered for a duration of about 5 days to 365 days, about 5 days to 300 days, about 5 days to 300 days, about 5 days to 250 days, about 5 days to 200 days, about 5 days to 100 days, about 5 days to 60 days, about days to 30 days, about 5 days to 14 days, or about 3 days to 7 days, preferably about 21 days to 28 days.

In some embodiments, the desired dosage of the formulations described herein can be administered once daily or multiple times in a day. For example, a treatment regimen can include administering a dosage level sufficient to deliver 10 mg/kg body weight twice daily, 20 mg/kg body weight twice daily, 50 mg/kg body weight once daily, 10 mg/kg body weight three times daily, 20 mg/kg body weight four times daily, or 50 mg/kg body weight twice daily.

### Pulse Dosing

In some embodiments, subjects can be administered a pulse dose of the therapeutic agents and/or pharmaceutical formulations of the present disclosure. As used herein, “pulse” refers to the plurality of doses at spaced apart time intervals. Generally, upon administration of the first dose, the growth of the bacteria can be inhibited, retarded and/or the bacteria can be killed. Following, the first dose, the bacteria levels can increase; and a second dose can be initiated. Eradication of bacteria can therefore be achieved by several rounds of pulse dosing. As a non-limiting example, the pulse dosing schedule can include a first round of dosing that includes administration of the pharmaceutical formulation for 5 days and allowing a period of recovery of about 24 hours followed by a second round of dosing. Additional rounds of dosing (e.g., third and fourth round of dosing) for 5 days each can be incorporated after 24-hour recovery period between each round of dosing. In some embodiments, pulse dosing schedules include pharmaceutical formulation administration from about every 2 hours to about every 10 hours, from about every 4 hours to about every 20 hours, from about every 6 hours to about every 30 hours, from about every 8 hours to about every 40 hours, from about every 10 hours to about every 50 hours, from about every 12 hours to about every 60 hours, from about every 14 hours to about every 70 hours, from about every 16 hours to about every 80 hours, from about every 18 hours to about every 90 hours, from about every 20 hours to about every 100 hours, from about every 22 hours to about every 120 hours, from about every 24 hours to about every 132 hours, from about every 30 hours to about every 144 hours, from about every 36 hours to about every 156 hours, from about every 48 hours to about every 168 hours, from about every 2 days to about every 10 days, from about every 4 days to about every 15 days, from about every 6 days to about every 20 days, from about every 8 days to about every 25 days, from about every 10 days to about every 30 days, from about every 12 days to about every 35 days, from about every 14 days to about every 40 days.

### VI. Definitions

Administer: The terms “administer,” “administration,” “administering,” and the like, when used in conjunction with a therapeutic agent means to deliver a therapeutic agent to a subject whereby the therapeutic agent positively impacts, i.e., has a therapeutic effect on, the subject or the tissue or the organ to which it is targeted. The therapeutic agents described herein can be administered either alone or in combination (concurrently or serially) and/or with other pharmaceuticals. For example, the therapeutic agents can be administered in combination with vaccines, antibiotics, antiviral agents, anti-cancer or anti-neoplastic agents, or in combination with other treatment modalities such as herbal therapy, acupuncture, naturopathy, etc.

Effective Amount: The term “effective amount” as used herein generally refers to an amount of the therapeutic agent that is administered to decrease, prevent or inhibit the disease. The amount will vary for each compound and upon known factors related to the item or use to which the therapeutic agent is applied.

Modulation: The term “modulation” refers to up regulation (i.e., activation or stimulation), down regulation (i.e., inhibition or suppression) of a response, or the two in combination or apart.

Pharmaceutically acceptable: The term “pharmaceutically acceptable”, as used herein, refers to compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the U.S. Food and Drug Administration. A “pharmaceutically acceptable carrier”, as used herein, refers to all components of a pharmaceutical formulation that facilitate the delivery of the composition in vivo. Pharmaceutically acceptable carriers include, but are not limited to, diluents, preservatives, binders, lubricants, disintegrators, swelling agents, fillers, stabilizers, and combinations thereof.

Subject: A “subject” can include a human subject for medical purposes, such as for the treatment of an existing disease, disorder, condition or the prophylactic for preventing the onset of a disease, disorder, or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal can be a transgenic animal. In some embodiments, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like).

Therapeutic agent: As used herein, the term “therapeutic agent” refers to any substance used to restore or promote the health and/or wellbeing of a subject and/or to treat, prevent, alleviate, cure, or diagnose a disease, disorder, or condition.

Treatment or Treating: The terms “treatment,” “treating,” and the like, refer to an intervention performed with the intention altering the pathology or symptoms of a disorder. In some embodiments, the treatment is for therapeutic treatment. Those in need of treatment can include those already with the disorder. In some embodiments, the treatment is for experimental treatment.

Preventing: The term “preventing” as used herein, refers to an intervention performed to decrease the chance of getting a disorder, disease or condition or a symptom associated with a disorder, disease or a condition.

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

The present disclosure is further illustrated by the following non-limiting examples.

### EXAMPLE

### Example 1. Experimental Overview: Assaying for Treponema denticola Sensitivity to Hygromycin A (Hyg A)

Hyg A sensitivity as determined by both minimum inhibitory concentration (MIC) and minimum bactericidal concentration (MBC) of a representative group of T. denticola strains including both “laboratory” and “clinical isolate” strains. The tested strains had levels of characterization varying from considerable to essentially none. For control and comparison purposes, Hyg A sensitivity of several oral bacterial species representative of the wide range of bacterial phyla present in the oral microbiome was tested in parallel. While essentially all oral microbes are naturally occurring “commensals”, several are more highly associated with disease (caries, gingivitis, periodontitis), particularly when present at elevated levels compared to what is commonly found in health. In the present study, the strains tested included Fusobacterium nucleatum (ATCC 25586), Parvimonas micra (ATCC 33270) and Porphyromonas gingivalis (ATCC 33277). The MIC and MBC of Hyg A against selected oral bacterial species other than Treponema was determined following the Clinical and Laboratory Standards Institute (CLSI) standards described by Wiegand et al., 2008 (“Agar and broth dilution methods to determine the minimal inhibitory concentration (MIC) of antimicrobial substances.” Nat Protoc 3:163-75. 10.1038/nprot.2007.521, incorporated herein by reference) with the modifications documented below. These strains were grown at 37° C. in standard Tryptone Yeast Extract medium with supplements as required for growth of individual species and under appropriate atmospheric conditions (anaerobic or 5% CO2).

The minimum inhibitory concentrations (MICs) and minimum bactericidal concentrations (MBCs) of hygromycin A (Hyg A) against Treponema denticola strains were determined. T. denticola was grown at 37° C. in an anaerobic chamber (Coy Laboratory Products, Grass Lake, MI) in an atmosphere consisting of nitrogen, hydrogen and carbon dioxide (ratio 85:10:5). Under these conditions, T. denticola generation time in liquid media is approximately 12 h. T. denticola was grown in deep 96-well polystyrene plates (ThermoScientific 278606; well volume 0.9 ml) with parafilm-sealed lids compared with growth in polystyrene tubes. T. denticola was grown under anaerobic conditions to late log phase (4 days) in TYGVS medium containing 2.5% heat-inactivated serum, then diluted 1:20 in 5 ml fresh complete TYGVS containing a range of Hyg A concentrations, and monitored for up to 6 days under anaerobic conditions to determine the MIC. A Hyg A concentration range between 0.01 μg/ml and 1.0 μg/ml was selected for initial studies. Controls were included in all experiments; specifically, no antimicrobial (negative control) and erythromycin (40 μg/ml; positive control). All experiments were conducted with triplicate samples, and each experiment was conducted twice.

### Example 2. MIC of Hygromycin a Against Oral Microbes

A used herein, Minimum Inhibitory Concentration (MIC) refers to the lowest concentration of antimicrobial that inhibits visible growth compared to the negative control, as indicated by a difference (p≤0.05) in optical density at 600 nm (OD₆₀₀).

Phenol red was used to monitor for T. denticola growth. T. denticola cultures were diluted 1:20 in TYGVS media containing phenol red (50 μg/ml) and a range of concentrations of Hyg A. The initial concentrations tested were 0, 0.01, 0.02, 0.05, 0.1, 0.2, 0.5, 1, 2, and 5 μg/ml. Kanamycin (100 μg/ml) was included as a positive control. Initial experiments with T. denticola 35405 were conducted in duplicate with and without phenol red in order to validate that visible growth in broth corresponded with phenol red color change from yellow to red (not shown). The assay was conducted in 96-well polystyrene plates in a total volume/well of 0.5 ml per well. Plates were sealed to prevent evaporation and incubated anaerobically for up to 14 days. Stable results were obtained with Treponema strains in 7-10 days. Incubation conditions and times varied depending on species. For example, S. salivarius was grown in 5% CO2 atmosphere for either 24 hours or 4 days. Extended incubation resulted in increased MIC for this species from 10 μg/ml to 20 μg/ml.

Following incubation, plates were photographed. To quantitate results, A562/A630 ratios were calculated for each well by transferring 0.1 ml culture supernatant to a flat-bottom 96-well plate and scanning in a Varioskan Flash plate reader (Thermo Scientific). Higher A562/A630 ratio values indicate growth inhibition.

### Preparation of Cultures for Antibiotic Testing

T. denticola cultures were grown from frozen stock in TYGVS broth medium. Bacteria other than T. denticola were grown from frozen stock on Brucella blood agar supplemented with hemin and Vitamin K (ThermoScientific BD297848). Cell integrity and morphology was verified by microscopy (400× dark field). OD₆₀₀ was measured for all cultures and culture volume required for 1/20 volume deep well plate cultures was calculated (900 μl final volume per well). For each set of triplicate samples, 18 ml final volume of [media+culture] was prepared such that OD₆₀₀ 0.45 culture was 1/10 of final volume (i.e., 1.8 ml of OD₆₀₀=0.45 culture+16.2 ml media) was adjusted appropriately. 450 μl antibiotic-containing indicator media (containing 100 μg/ml phenol red) was added to appropriate wells of deep 96-well plate. After incubation, absorbance was measured at A₅₆₂, and A₆₃₀.

P. gingivalis is unable to grow at acidic pH, and thus no phenol red color change could be detected. Growth was therefore monitored, and MIC determined by both changes in OD₆₀₀ values and by dark field microscopy and by comparing cells/field at inoculation and after incubation for several days.

### Example 3. Determining MBC of Hygromycin A Against T. denticola and Other Oral Microbes

For determination of MBC, 0.1 ml of bacterial samples were removed from tubes that have antimicrobial concentrations equivalent to and higher than the MIC, and cultured in the absence of antimicrobial in TYGVS broth medium or serially diluted and plated on TYGVS-Noble agar plates. To isolate individual colonies and/or to determine CFU T. denticola was plated at appropriate dilutions in a low-melting-point agar formulation and incubated for 1-2 weeks until subsurface colonies formed. The MBC was defined as the lowest concentration of Hyg A that killed at least 99.9% of the bacteria in a given time. An MBC/MIC ratio of >4 indicates a bacteriostatic effect, while a MBC/MIC ratio of <4 indicates a bactericidal effect (see Pankey et al. 2004. Clin Infect Dis 38:864-70. 10.1086/381972; the contents of which are herein incorporated by reference in its entirety).

For T denticola, a single 24 well plate with 2 ml/well TYGVS 0.8% noble agar+supplements of 5% HIHS, rifampicin 4 ug/ml, fosfomicin 100 μg/ml, was prepared. 5 μl culture from the bottom of each well of the deep 96 well MIC plate was stabbed into solid media of 24 well plate. The plate was incubated for at least a week (37° C., anaerobic) and the presence or absence of growth was recorded. For P. micra, S salivarius, F. nucleatum, and P. gingivalis, 3-5 μl of the culture was spotted on a Brucella blood agar plate plus hemin and vitamin K from each triplicate well at concentrations of MIC, above and/or below MIC. The cultures were incubated at 37° C. (anaerobic or 5% CO2 as appropriate) and the presence or absence of growth was recorded.

Table 1 below summarizes the MIC and MBC results as well as the ratio of MIC to MBC.

TABLE 1
MIC and MBC results

| | Hyg A | MIC (μg/ml) | | MBC (μg/ml) | | MIC:MBC |
|---|---|---|---|---|---|---|
| Strain | (μg/ml) | 1 | 2 | 1 | 2 | ratio |
| T. denticola 35405 | 0.01-5.0 | 0.2 | 0.2 | 1.0 | 0.5 | 0.2-0.4 |
| T. denticola 35404 | 0.01-5.0 | 0.5-1.0 | 5.0 | 5.0 | >5.0 | 0.1-1.0 |
| T. denticola 33520 | 0.01-5.0 | 1.0 | 1.0 | 5.0 | 1.0 | 0.2-1.0 |
| T. denticola 33521 | 0.01-5.0 | 0.5 | 2.0 | 0.5 | 2.0 | 0.3-1.0 |
| T. denticola OTK | 0.01-5.0 | 0.5 | 1.0 | 1.0 | 1.0 | 0.5-1.0 |
| T. denticola H1-T | 0.01-5.0 | 0.5 | 0.5 | 0.5 | 0.5 | 1.0 |
| T. denticola SP44 | 0.01-5.0 | 1.0 | 1.0 | 5.0 | 2.0 | 0.5-2.0 |
| T. denticola ASLM | 0.01-5.0 | 0.5 | 1.0 | 1.0 | 1.0 | 0.5-1.0 |
| T. denticola US Trep | 0.01-5.0 | 0.5 | 0.5 | 1.0 | 1.0 | 0.5 |
| S. salivarius 13419 | 0.1-50 | 10 | 10 | 10 | 10 | 10 |
| F. nucleatum 25586 | 0.1-50 | 1.0 | 1.0 | 1.0 | 1.0 | 1.0 |
| P. micra 33270 | 0.5-200 | 2.0 | 2.0 | >10 | >>10 | >0.2 |
| P. gingivalis 33277 | 0.01-5.0 | 5.0 | 5.0 | >5.0 | >5.0 | >1.0 |

This study revealed that MIC values for Treponema denticola strains were fairly consistently in the range of 0.5 μg/ml (0.2-1.0 μg/ml). The Type strain (ATCC 35405) was the most sensitive (MIC=0.2 μg/ml). Another ATCC strain (35404) was less sensitive (0.5-5 μg/ml). Hyg A sensitivity of low passage clinical isolates varied from 0.2 μg/ml (strain ASLM) to 1 μg/ml (strain SP44). There was no apparent bias between established laboratory strains and relatively low-passage clinical isolates. Interestingly, ATCC 35404 was the strain most tolerant of Hyg A (MIC and MBC=5 μg/ml). The ratio of MBC/MIC for all T. denticola strains averaged 2.3, suggesting that the effects of Hyg A are bactericidal.

Most of the other oral bacterial species tested were markedly more resistant than T. denticola to effects of Hyg A. In general, Gram-positive strains were more tolerant of Hyg A than were Gram-negatives. Fusobacterium nucleatum ATCC 25586 was the most sensitive, at approximately 1 μg/ml (MIC and MBC). Overall, Streptococcus salivarius ATCC13419 was the most tolerant, at 10 μg/ml (MIC and MBC). Parvimonas micra ATCC 33270 had a relatively low MIC value of 2 μg/ml, yet its MBC value was >10 μg/ml. Relatively little is known regarding the metabolism of this Gram-positive anaerobe. These data suggest that Hyg A was bacteriostatic against P. micra and bactericidal against the other strains tested.

### Example 4. Kirby-Bauer Diffusion Assay on Fusobacterium nucleatum Strains

Three representatives of 6 subspecies of Fusobacterium nucleatum (F. nucleatum) for a total of 18 strains were tested in the experiment. The sub species and strains are described in Table 2.

TABLE 2
F. nucleatum strains

| Sub species | Strain | Description |
|---|---|---|
| F. nucleatum | 7/1 (OR 7_1) | Invasive isolate from a Crohn's Disease |
| animalis | | lesion |
| | CRC 7/3 JVN3C1 (OR | Invasive isolate from CRC tumor tissue |
| | CRC 7_3JVN3C1) | |
| | 218A8 | Isolate from oral cancer lesion |
| F. nucleatum | 215A9 | Isolate from oral cancer lesion |
| vincentii | CC53 | Non-invasive isolate from CRC tumor |
| | | tissue |
| | 3/1/36A2 (EAV018) | Invasive isolate from a Crohn's Disease |
| | | lesion |
| F. nucleatum | ATCC25586T | Type strain |
| nucleatum | 203L34 | Isolate from oral cancer lesion |
| | 2/3 FMU 1 (2_3 FMU 1) | Isolate from CRC tumor tissue |
| F. nucleatum | 203L28 | Isolate from oral cancer lesion |
| polymorphum | 203L29 | Isolate from oral cancer lesion |
| | 13/3C (13_3C EAV005 | Invasive isolate from a Crohn's Disease |
| | OR EAVG 005) | lesion |
| F. nucleatum | 203C15 | Isolate from oral cancer patient, off- |
| fusiforme | | lesion |
| | 203L25 | Isolate from oral cancer lesion |
| | 203L30 | Isolate from oral cancer lesion |
| F. nucleatum | 2/1/31 OR (2_1_31 | Invasive isolate from a Crohn's Disease |
| periodonticum | EAV015 OR EAVG 015) | lesion |
| | 3/1/7B (3_1_7B OR | Invasive isolate from ulcerative colitis |
| | EAVG 011) | lesion |
| | 209B32 | Isolate from oral cancer patient, off- |
| | | lesion |

All strains were verified by 16S V3/V6 sequence analysis prior to use. The methods described herein were performed in an anaerobic chamber at 37° C. All media and plates were degassed prior to use.

Hygromycin A (Hyg A) discs were prepared by solubilizing Hyg A in distilled water and sterile filtered. Clindamycin was used as a positive control. Defined amounts of hygromycin A (40, 20, 10 or 5 micrograms), clindamycin (2 micrograms) or sterile distilled water were absorbed onto Kirby-Bauer discs in a biosafety cabinet.

An overnight culture was grown in Wilkins-Chalgren (WC) broth and back-diluted 1:10 in fresh broth. When an OD 600 reading reached 0.5, 300 μL of the log phase culture was plated onto WC agar plates. The plates were sectioned into quadrants and a Kirby-Bauer (KB) disc impregnated with a defined amount of hygromycin A was placed in each quadrant. Plates were prepared thus in triplicate and the four amounts of hygromycin A were 40, 20 10 and 5 micrograms. The same volume of culture was spread on fourth plate sectioned into six sections for three positive and three negative controls. KB discs with water served as negative controls and KB discs impregnated with 2 micrograms of clindamycin served as positive controls. Plates were incubated for 24-48 hours and the zones of inhibition of growth measured. Where inhibition of strain was too large to measure (i.e. greater than the plate would allow), these replicates were assigned the maximum value of 42 mm. Table 3, Table 4, Table 5, Table 6, Table 7, Table 8 show the mean size of zone inhibitions for each of the subspecies tested using increasing concentrations of hygromycin A where the standard error of mean is represented in parenthesis.

TABLE 3
F. nucleatum vincentii size of zone inhibition (mm)

| Hyg A | | | |
|---|---|---|---|
| Concentration (μg) | 215A9 | 3/1/36A2 | CC53 |
| 5 | 0.00 | 19.00 | 16.33 (±0.67) |
| 10 | 0.00 | 21.33 (±0.88) | 21.33 (±1.33) |
| 20 | 0.00 | 24.67(±0.33) | 24.00 (±2.08) |
| 40 | 0.00 | 27.67(±0.33) | 27.33 (±1.77) |

TABLE 4
F. nucleatum animalis size of zone inhibition (mm)

| Hyg A | | | CRC 7_3 |
|---|---|---|---|
| Concentration (μg) | 218A8 | 7_1 | JVN3C1 |
| 5 | 19.67 (±0.33) | 18.33 (±0.88) | 0.00 |
| 10 | 21.67 (±0.88 | 18.00 (±1.00) | 0.00 |
| 20 | 29.67 (±0.33 | 22.67 (±1.45) | 0.00 |
| 40 | 42.00 (±0.00) | 24.33 (±0.33) | 0.00 |

TABLE 5
F. nucleatum fusiform size of zone inhibition (mm)

| Hyg A | | | |
|---|---|---|---|
| Concentration (μg) | 203C15 | 203L25 | 203L30 |
| 5 | 18.00 | 25.00 | 22.33 (±0.33) |
| 10 | 24.00 | 28.67 (±0.67) | 24.33 (±0.33) |
| 20 | 42.00 | 42.00 | 28.00 (±1.16) |
| 40 | 42.00 | 42.00 | 30.00 |

TABLE 6
F. nucleatum nucleatum size of zone inhibition (mm)

| Hyg A | | | |
|---|---|---|---|
| Concentration (μg) | 25586 | 25586 | 203L34 |
| 5 | 16.00 | 25.67 (±0.67) | 24.00 (±1.53) |
| 10 | 20.00 | 30.00 (±0.58) | 30.00 (±1.16) |
| 20 | 26.00 | 42.00 | 39.67 (±2.34) |
| 40 | 30.00 | 42.00 | 42.00 |

TABLE 7
F. nucleatum periodonticum size of zone inhibition (mm)

| Hyg A | 2_1_31 | | |
|---|---|---|---|
| Concentration (μg) | EAV015 | 209B32 | 3_1_7B |
| 5 | 20.33 (±0.88) | 22.00 (±1.00) | 29.33 (±0.33) |
| 10 | 23.33 (±0.88) | 25.67 (±0.67) | 34.33 (±0.33) |
| 20 | 25.33 (±0.33) | 30.67 (±0.67) | 36.00 (±1.00) |
| 40 | 30.00 | 35.00 | 40.00 (±1.16) |

TABLE 8
F. nucleatum polymorphum size of zone inhibition (mm)

| Hyg A Concentration | 13_3C | | |
|---|---|---|---|
| (μg) | EAV005 | 203L28 | 203L29 |
| 5 | 16.67 (±0.33) | 20.33 (±0.33) | 18.33 (±0.33) |
| 10 | 20.00 (±1.16) | 23.00 (±2.52) | 20.33 (±0.33) |
| 20 | 25.67 (±0.67) | 24.00 | 24.00 (±0.58) |
| 40 | 27.33 (±0.67) | 30.33 (±0.33) | 26.67 (±1.20) |

The amount of hygromycin A was plotted against the size of the zone of inhibition for each strain tested. The results are shown in Table 9. In Table 9, N/A indicates fully resistant strains for which R² values were not determined ((CRC 7_3JVN3C1 and 215A9). A line of best fit was applied to each plot. A linear regression was then performed to find the line of best fit and R² values which are statistical measures used to compare the data to the line of best fit. A value of 1 indicates a perfect fit, with lower values indicating less than a perfect fit. The lowest R² lowest value calculated was 0.72, with most values being considerably higher. These data show that the relationship between hygromycin A resistance and strain sensitivity is linear for the range of hygromycin A concentrations.

TABLE 9
Regression values for the relationship of the zone of
inhibition (in mm) versus the amount of hygromycin A

| Subtype | Strain | Slope | Intercept | R² |
|---|---|---|---|---|
| F. nucleatum | 218A8 | 0.65 | 16.00 | 1.00 |
| animalis | 7_1 | 0.19 | 17.29 | 0.86 |
| | CRC 7_3JVN3C1 | 0 | 0 | NA |
| F. nucleatum | 203C15 | 0.68 | 18.78 | 0.72 |
| fusiforme | 203L25 | 0.49 | 25.28 | 0.72 |
| | 203L30 | 0.21 | 22.19 | 0.90 |
| | 25586 | 0.38 | 15.83 | 0.91 |
| F. nucleatum | 2_3 FMU 1 | 0.46 | 26.32 | 0.72 |
| nucleatum | 203L34 | 0.49 | 24.80 | 0.80 |
| F. nucleatum | 2_1_31 EAV015 | 0.26 | 19.91 | 0.97 |
| periodonticum | 209B32 | 0.35 | 21.68 | 0.93 |
| | 3_1_7B | 0.27 | 29.93 | 0.87 |
| F. nucleatum | 13_3C EAV005 | 0.29 | 16.97 | 0.82 |
| polymorphum | 203L28 | 0.27 | 19.36 | 0.97 |
| | 203L29 | 0.23 | 17.99 | 0.93 |
| F. nucleatum | 215A9 | 0 | 0 | NA |
| vincentii | 3/1/36A2 | 0.24 | 18.71 | 0.94 |
| | CC53 | 0.28 | 17.04 | 0.86 |

3 strains were tested from each subspecies. Among the strains tested, a trend towards strains of F. nucleatum vincentii, F. nucleatum polymorphum and F. nucleatum periodonticum being more resistant to hygromycin A compared to other F. nucleatum strains was observed. Whereas F. nucleatum nucleatum and F. nucleatum fusiforme showed a trend towards being more sensitive. The resistance of F. nucleatum animalis strain CRC 7_3JVN3C1 and F. nucleatum vincentii strain 215A9 out of all the strains tested was unexpected, given the sensitivity of F. nucleatum strain 25586 to Hygromycin A shown in example 3.

### Example 5. Susceptibility of Treponema pallidum pallidum

Efficacy of hygromycin A against Treponema pallidum pallidum strains was tested by co-culturing the pathogen with rabbit epithelial cell.

The SS14 and Chicago strain of T. pallidum were obtained from frozen stocks of treponemes propagated intratesticularly (IT) in a New Zealand White rabbit (Oryctolagus cuniculus) as previously reported by Baker-Zander S. A. et al. (J Immunol. 1988; 141(12):4363-9; the contents of which are herein incorporated by reference in its entirety).

The T. pallidum culture system was set up to perform susceptibility tests and modified from the culture system previously described by Edmondson et al. (mBio. 2018; 9(3). Epub 2018/06/28; the contents of which are herein incorporated by reference). After passaging treponemes in six-well culture plates (Corning Inc, Corning, NY), the cells were sub-cultured into two 96-well cell culture plates to allow a total of 8 replicates for each antibiotic concentration to be tested. The day before inoculation, three 96-well cell culture plates were seeded with 3×10³ rabbit Sf1Ep cells per well in 150 μL of culture media. The plates were then incubated overnight in a 5% CO₂ atmosphere within a Hera Cell 150 incubator (Thermo Fisher Scientific) to facilitate cellular adhesion to the plate surface. On the same day, TpCM-2 media was prepared as described by Edmondson et al (mBio. 2018; 9(3). Epub 2018 Jun. 28). The TpCM-2 media was equilibrated overnight at 34° C. in a microaerophilic environment consisting of 1.5% O₂, 3.5% CO₂ and 95% N₂ supplied as a tri-gas mix (Praxair, Danbury, CT) in a tri-gas incubator (Hera Cell VIOS 160i, Thermo Fisher). The following day, cell culture media was removed from the 96-well plates, and cells were rinsed with equilibrated TpCM-2 media. Subsequently, each well was filled with 150 μL of equilibrated TpCM-2 media and the plate was transferred to the tri-gas incubator to be equilibrated in the microaerophilic atmosphere for not less than three hours. To prepare the treponemal inoculum for the 96-well plates, the Sf1Ep cells inoculated the previous week with T. pallidum were trypsinized to allow the release and enumeration of spirochetes. Treponemes were counted using dark field microscopy on a Leica DM2500 LED microscope (Leica, Wetzlar, Germany) and diluted in TpCM-2 to 2.0×10⁴ T. pallidum cells/ml. To obtain a treponemal inoculum of 3×10³ cells, 150 μL were added to each well of the 96-well plates. Two antibiotics were tested in this study: Hygromycin A, at different concentrations (0.03, 0.06, 0.12, 0.24, 0.48, 0.96, 1.92, 3.84 μg/mL), and penicillin G (a known treponemicidal antibiotic) at 60 ng/ml. Each concentration was tested in 8 replicates. To minimize volume variation, each antibiotic solution was added from a 100× concentrated stock to achieve the final concentration to be tested. No-antibiotic wells, as well as solvent-only wells, were also included as controls. After seeding of the treponemes and addition of the antibiotics, the culture plates were incubated at 34° C. for seven days in tri-gas incubator.

Following the seven-day incubation period, two of the four plates were processed for DNA extraction, while the remaining two plates were used to seed a no-antibiotic control plate prepared the previous day as described above. For DNA extraction, the plates were removed from the incubator and the culture media was removed with a multichannel pipet from each well and discarded. Cells were not trypsinized but directly mixed with 200 μL of Genomic Lysis Buffer (Zymo Research, Irvine, CA) for DNA extraction and incubated for 30 min at room temperature to allow cellular lysis to complete. Following cell lysis, the plates were frozen at −20° C. until extraction.

To purify DNA, the 96-well plates were thawed at 56° C. in a dry incubator and quickly spun to recuperate condensation drops on the well lids. DNA was extracted using a Quick DNA-96 kit (Zymo Research) according to the manufacturer's protocol. DNA was eluted in 100 μl molecular grade water (Sigma-Aldrich) and stored at −20° C. until amplification. The other two 96-well plate were processed to inoculate a new one containing Sf1Ep cells but no antibiotics in the culture media to confirm treponemicidal activity of the tested drugs in each strain tested. The culture media was removed from the third 96-well plate and 20 μl of trypsin were added to detach Sp1Ep and treponemes. Half of the resulting suspension was inoculated into a new 96-well plate without antibiotic (prepared as described above), which was then incubated for seven more days. After a week, the no-antibiotic plate was processed to extract DNA and evaluate treponemal load by qPCR.

Following DNA extraction, treponemal burden was evaluated for each sample in triplicate using a qPCR approach targeting the tp0574 gene (also called T47) previously described (see Giacani L, et al. Infect Immun. 2007; 75(1):104-12; the contents of which are herein incorporated by reference in its entirety). A 313 bp amplicon was generated by qPCR with an 89° C. melting temperature. Briefly, an absolute quantification protocol using an external standard was used to quantify the tp0574 gene copy number at the time of sample harvest. Amplifications were run on a QuantStudio 5 thermal cycler (Thermo Fisher Scientific) and results analyzed using the instrument software. Data were imported into Prism 8 (GraphPad Software, San Diego, CA).

Cytotoxicity assay was performed according to the Cell Proliferation Reagent WST-1 protocol (Attached; Roche, Basel, Switzerland). Absorbance was measured after 1 hour incubation with the reagent. No significant differences were found between day 7 cells incubated without antibiotic and day 7 cells incubated with increasing concentrations of hygromycin A. WST-1 assay showed that hygromycin A was not toxic to SF1Ep cells at any concentration tested. This result supports the conclusion that the outcome of the susceptibility assay is solely due to the effect of the drug on the treponemal cells.

Susceptibility assay supports that hygromycin A was effective against the Chicago strain up to 120 ng/ml (0.12 μg/ml). Significantly higher growth (p<0.05) was detected at 0.06 μg/ml when compared to higher concentrations of hygromycin A. No significant difference was found between 0.12 μg/ml and higher concentrations of hygromycin A. Analysis was performed using ANOVA with significance set at p<0.05. The data are shown in Table 10.

Table 11 shows effects of hygromycin A on Chicago strain seeded onto a no-antibiotic control plate. These studies confirmed the treponemicidal activity of hygromycin A at 0.12 μg/ml or higher. At these concentrations, hygromycin A was as effective as penicillin G (treponemicidal at 60 ng/ml). As expected, growth was detected at 0.06 μg/ml, based on the results shown in Table 10. In Table 10 and Table 11, Ctrl means control samples, avg indicates average values and Pen indicates penicillin.

TABLE 10
Mean T47 copies/μL in Chicago C strain

| | Experimental values | | | | | | | | | Avg. |
|---|---|---|---|---|---|---|---|---|---|---|
| Ctrl | Day 0 | 511 | 648 | 595 | 591 | 399 | 743 | 471 | 791 | 593 |
| | Day 4 | 3381 | 3741 | 5431 | 4502 | 3106 | 5780 | 5243 | 4055 | 4405 |
| | Day 7 | 6612 | 12552 | 12496 | 19398 | 17433 | 17786 | 14741 | 9307 | 13791 |
| Hyg A- | 3.84 | 324 | 470 | 612 | 487 | 485 | 532 | 618 | 412 | 493 |
| μg/ml | 1.92 | 301 | 422 | 453 | 556 | 585 | 546 | 464 | 279 | 451 |
| | 0.96 | 271 | 393 | 520 | 528 | 402 | 418 | 372 | 273 | 397 |
| | 0.48 | 341 | 475 | 462 | 614 | 548 | 509 | 425 | 280 | 457 |
| | 0.24 | 360 | 447 | 513 | 890 | 775 | 636 | 565 | 424 | 576 |
| | 0.12 | 449 | 775 | 689 | 776 | 788 | 670 | 704 | 545 | 674 |
| | 0.06 | 1336 | 1882 | 1716 | 2225 | 1815 | 2044 | 2022 | 1476 | 1814 |
| | 0.03 | 2531 | 7304 | 8369 | 7552 | 6189 | 9263 | 8429 | 7834 | 7184 |
| Ctrl | Carrier | 7387 | 16196 | 20165 | 19807 | 19016 | 18648 | 17464 | 4605 | 15411 |
| Ctrl | 60 ng/ml | 294 | 226 | 304 | 137 | 328 | 261 | 222 | 241 | 252 |
| | Pen | | | | | | | | | |

TABLE 11
Mean T47 copies/μL of Chicago C strain seeded on no antibiotic plate

| Chicago | | Hygromycin A (μg/ml) | | | | | | | | Carrier | 60 ng/ml |
|---|---|---|---|---|---|---|---|---|---|---|---|
| C | Day 7 | 3.84 | 1.92 | 0.96 | 0.48 | 0.24 | 0.12 | 0.06 | 0.03 | only | Pen. |
| Expt. | 11382 | 13 | 34 | 7 | 22 | 37 | 9 | 50 | 5586 | 15556 | 15 |
| values | 26329 | 18 | 13 | 13 | 43 | 13 | 48 | 908 | 14839 | 44533 | 11 |
| | 32748 | 23 | 13 | 18 | 37 | 27 | 35 | 1184 | 19191 | 42524 | 22 |
| | 36947 | 30 | 31 | 60 | 23 | 48 | 42 | 1573 | 17902 | 41823 | 12 |
| | 42889 | 37 | 28 | 14 | 0 | 17 | 13 | 744 | 10276 | 10501 | 12 |
| | 30574 | 24 | 8 | 23 | 32 | 41 | 19 | 560 | 18338 | 43756 | 41 |
| | 25765 | 27 | 23 | 31 | 32 | 22 | 39 | 2087 | 20335 | 6454 | 74 |
| | 9798 | 41 | 36 | 27 | 23 | 52 | 22 | 892 | 9952 | 12884 | 22 |
| Avg. | 27054 | 27 | 23 | 24 | 26 | 32 | 28 | 1000 | 14552 | 27254 | 26 |

Susceptibility assay using S S14 strain also supported the efficacy of hygromycin A against SS14 up to 1.92 μg/ml. Growth, although minimal, was detected at all lower concentrations. No significant difference was found between 1.92 μg/ml and higher concentrations of hygromycin A. Analysis was performed using ANOVA with significance set at p<0.05. The results are shown in Table 12.

SS14 strain seeded onto a no-antibiotic control plate confirmed treponemicidal activity of hygromycin A at 0.12 μg/ml or higher. At these concentrations, hygromycin A was as effective as penicillin G (treponemicidal at 60 ng/ml). The results are shown in Table 13. Growth was detected at 0.06 μg/ml, as expected based on the results shown in Table 12. In Table 12 and Table 13, Ctrl means control samples, avg indicates average values and Pen indicates penicillin.

TABLE 12
Mean T47 copies/μL of SS14 C strain

| | Experimental values | | | | | | | | | Average |
|---|---|---|---|---|---|---|---|---|---|---|
| Ctrl. | Day 0 | 308 | 373 | 327 | 412 | 356 | 639 | 475 | 482 | 422 |
| | Day 4 | 1129 | 1956 | 2091 | 2503 | 2240 | 2591 | 1923 | 1509 | 1993 |
| | Day 7 | 2547 | 5211 | 5929 | 6786 | 7743 | 7102 | 6787 | 3427 | 5691 |
| Hyg A | 3.84 | 164 | 228 | 360 | 281 | 409 | 264 | 310 | 185 | 275 |
| μg/ml | 1.92 | 358 | 405 | 363 | 328 | 285 | 387 | 294 | 166 | 323 |
| | 0.96 | 150 | 307 | 435 | 361 | 405 | 327 | 299 | 268 | 319 |
| | 0.48 | 327 | 435 | 503 | 508 | 487 | 453 | 342 | 352 | 426 |
| | 0.24 | 510 | 491 | 572 | 587 | 564 | 600 | 441 | 360 | 516 |
| | 0.12 | 372 | 629 | 710 | 787 | 926 | 951 | 965 | 552 | 737 |
| | 0.06 | 713 | 883 | 851 | 1260 | 984 | 888 | 837 | 911 | 916 |
| | 0.03 | 1524 | 2877 | 2662 | 2752 | 3084 | 3282 | 3342 | 2625 | 2768 |
| Ctrl | Carrier | 2175 | 4388 | 5314 | 6389 | 6700 | 6812 | 5120 | 2530 | 4928 |
| | only | | | | | | | | | |
| Ctrl | 60 ng/ml | 189 | 200 | 166 | 282 | 184 | 223 | 214 | 271 | 216 |
| | Pen. | | | | | | | | | |

TABLE 13
Mean T47 copies/μL of SS14 strain seeded on no antibiotic plate

| | | Hygromycin A (μg/ml) | | | | | | | | Carrier | 60 ng/ml |
|---|---|---|---|---|---|---|---|---|---|---|---|
| SS14 | Day 7 | 3.84 | 1.92 | 0.96 | 0.48 | 0.24 | 0.12 | 0.06 | 0.03 | only | Pen. |
| Expt. | 1610 | 7 | 8 | 16 | 31 | 5 | 22 | 71 | 445 | 1597 | 13 |
| values | 4649 | 15 | 5 | 0 | 5 | 21 | 25 | 159 | 1541 | 4412 | 21 |
| | 4240 | 21 | 19 | 3 | 6 | 7 | 15 | 200 | 2079 | 7700 | 27 |
| | 7009 | 22 | 22 | 13 | 0 | 0 | 24 | 343 | 2511 | 5404 | 15 |
| | 8792 | 19 | 5 | 8 | 2 | 35 | 17 | 137 | 1649 | 3804 | 21 |
| | 7806 | 25 | 7 | 11 | 0 | 16 | 14 | 310 | 3690 | 8355 | 17 |
| | 7320 | 21 | 17 | 0 | 16 | 16 | 45 | 274 | 3831 | 6241 | 24 |
| | 2430 | 18 | 19 | 3 | 10 | 6 | 0 | 99 | 2114 | 2068 | 10 |
| Avg. | 5482 | 19 | 13 | 7 | 9 | 13 | 20 | 199 | 2232 | 4948 | 18 |

Taken together, these data show that hygromycin A at a concentration of 0.12 μg/ml or more has a treponemicidal effect.

### EQUIVALENTS AND SCOPE

Those skilled in the art will recognize, or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” can mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process. As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

It is also noted that the term “comprising” is intended to be open and permits but does not require the inclusion of additional elements or steps. When the term “comprising” is used herein, the term “consisting of” is thus also encompassed and disclosed. All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art can be explicitly excluded from any one or more of the claims. Since such embodiments are deemed to be known to one of ordinary skill in the art, they can be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes can be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

The compositions and methods are more particularly described below and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above.

What is claimed is:

1. A method for treating an oral infection of Treponema denticola, Fusobacterium nucleatum, Parvimonas micra, Porphyromonas gingivalis in a subject, comprising administering hygromycin A to the subject.

2. The method of claim 1, wherein, the Hygromycin A inhibits the growth of or kills the Treponema denticola, Fusobacterium nucleatum, Parvimonas micra, Porphyromonas gingivalis and does not inhibit the growth of or kill at least one species of beneficial oral or gut bacteria.

3. The method of claim 1, wherein a concentration of hygromycin A is from about 0.01 μg/ml to about 100 μg/ml.

4. The method of claim 3, wherein a concentration of hygromycin A is from about 0.01 μg/ml to about 1.0 μg/ml.

5. The method of claim 4, wherein a concentration of hygromycin A is 0.05 μg/ml.

6. The method of claim 4, wherein a concentration of hygromycin A is 0.1 μg/ml.

7. The method of claim 4, wherein a concentration of hygromycin A is 0.2 μg/ml.

8. The method of claim 4, wherein a concentration of hygromycin A is 0.5 μg/ml.

9. The method of claim 9, wherein a concentration of hygromycin A is 1.0 μg/ml.

10. The method of claim 4, wherein a concentration of hygromycin A is 2 μg/ml.

11. The method of claim 4, wherein a concentration of hygromycin A is 5 μg/ml.

12. The method of claim 1, wherein the subject has an oral disease, a systemic disease, a cancer, or a veterinary disease.

13. The method of claim 12, wherein the disease oral disease is periodontitis or gingivitis.

14. The method of claim 13, wherein the periodontitis is localized aggressive periodontitis, generalized aggressive periodontitis, pulp necrosis or periapical periodontitis.

15. The method of claim 12, wherein the cancer is colorectal cancer, gastric cancer or esophageal cancer.

16. The method of claim 1, wherein the hygromycin A is administered to the subject by an oral route or a parenteral route.

17. The method of claim 16, wherein the parenteral route is a subcutaneous route, an intravenous route, an intramuscular route, an intraperitoneal route, a transdermal route, an ocular route, or an inhalation route.

18. A method for treating a Fusobacterium nucleatum infection in a subject, comprising administering
hygromycin A to the subject.

19. The method of claim 18, wherein the hygromycin A inhibits the growth of or kills the Fusobacterium nucleatum but does not inhibit the growth of or kill at least one species of beneficial oral or gut bacteria.

20. The method of claim 18, wherein a concentration of hygromycin A is from about 1 μg/ml to about 100 μg/ml.

21. The method of claim 20, wherein a concentration of hygromycin A is 5 μg/ml.

22. The method of claim 20, wherein a concentration of hygromycin A is 10 μg/ml.

23. The method of claim 20, wherein a concentration of hygromycin A is 20 μg/ml.

24. The method of claim 20, wherein a concentration of hygromycin A is 40 μg/ml.

25. The method of claim 18, wherein F. nucleatum is one or more of F. nucleatum animalis, F. nucleatum vincentii, F. nucleatum nucleatum, F. nucleatum polymorphum, F. nucleatum fusiforme, or F. nucleatum periodonticum.

26. The method of claim 18, wherein the hygromycin A is administered to the subject by an oral route or a parenteral route.

27. The method of claim 26, wherein the parenteral route is a subcutaneous route, an intravenous route, an intramuscular route, an intraperitoneal route, a transdermal route, an ocular route, or an inhalation route.

28. A method for treating a cancer, a gastrointestinal disorder, or an oral disease in a subject, comprising contacting the subject with hygromycin A.

29. The method of claim 28, wherein the cancer, gastrointestinal disorder, or oral disease is associated with F. nucleatum infection.

30. The method of claim 29, wherein the cancer is colorectal cancer or oral cancer.

31. The method of claim 29, wherein the gastrointestinal disease is inflammatory bowel disease, Crohn's disease, ulcerative colitis, or colorectal cancer.

32. The method of claim 29, wherein the oral disease is a periodontal disease.

33. The method of claim 32, wherein the periodontal disease is chronic periodontitis, localized aggressive periodontitis, generalized aggressive periodontitis, pulp necrosis, or periapical periodontitis.

34. The method of claim 28, wherein the hygromycin A is administered at a concentration of from about 1 μg/ml to about 100 μg/ml.

35. The method of claim 34, wherein a concentration of hygromycin A is 5 μg/ml.

36. The method of claim 34, wherein a concentration of hygromycin A is 10 μg/ml.

37. The method of claim 34, wherein a concentration of hygromycin A is 20 μg/ml.

38. The method of claim 34, wherein a concentration of hygromycin A is 40 μg/ml.

39. The method of claim 28, wherein the hygromycin A is administered to the subject by an oral route or a parenteral route.

40. The method of claim 39, wherein the parenteral route is a subcutaneous route, an intravenous route, an intramuscular route, an intraperitoneal route, a transdermal route, an ocular route, or an inhalation route.

41. A method for treating a treponematoses infection in a subject comprising administering hygromycin A to the subject.

42. The method of claim 41, wherein the treponematoses infection is caused by Treponema pallidum or Treponema carateum infection.

43. The method of claim 42, wherein the hygromycin A inhibits the growth of or kills the Treponema pallidum or Treponema carateum but does not inhibit the growth of or kill at least one species of beneficial oral or gut bacteria.

44. The method of claim 43, wherein the treponematoses is syphilis, yaws, bejel or pinta.

45. The method of claim 41, wherein a concentration of hygromycin A is from about 0.01 μg/ml to about 100 μg/ml.

46. The method of claim 45, wherein a concentration of hygromycin A is from about 0.01 μg/ml to about 10 μg/ml.

47. The method of claim 46, wherein a concentration of hygromycin A is 0.06 μg/ml.

48. The method of claim 46, wherein a concentration of hygromycin A is 0.12 μg/ml.

49. The method of claim 46, wherein a concentration of hygromycin A is 0.24 μg/ml.

50. The method of claim 46, wherein a concentration of hygromycin A is 0.48 μg/ml.

51. The method of claim 46, wherein a concentration of hygromycin A is 0.96 μg/ml.

52. The method of claim 46, wherein a concentration of hygromycin A is 1.92 μg/ml.

53. The method of claim 46, wherein a concentration of hygromycin A is 3.84 μg/ml.

54. The method of claim 41, wherein the hygromycin A is administered to the subject by an oral route or a parenteral route.

55. The method of claim 54, wherein the parenteral route is a subcutaneous route, an intravenous route, an intramuscular route, an intraperitoneal route, a transdermal route, an ocular route, or an inhalation route.
